(19) United States
(12) Patent Application Publication
(10) Pub. No.: US 2004/0220538 A1
(43) Pub. Date: Nov. 4, 2004

(54) Hygienic diaper, sensor pad, and or sensing belt with alert, readout, transmission, paging, software & patient information database recording means for treating & caring for wetness, feces, & disease

(76) Inventor: Peter John Panopoulos, Hickory Hills, IL (US)

Correspondence Address: PETER JOHN PANOPOULOS, 9220 S. 87TH COURT, HICKORY HILLS, IL, 60457, US

(21) Appl. No.: 10/805,370

(22) Filed: Mar. 20, 2004

Related U.S. Application Data
(60) Provisional application No. 60/464,420, filed on Apr. 22, 2003.

(51) Int. Cl. A61F 13/15; A61F 13/20

(52) U.S. Cl. 604/361

(57) ABSTRACT

“Hygienic Diaper or Sensor Pad With Alert For Treating, Caring For Wetness, Feces, & Disease”; with one or more “sensors” detecting diseases, ketone levels, sugar levels, blood, body fluids, liquids, gasses, drugs, bacteria, viruses, toxins or other agents; a powered “circuit card unit” and transmission circuitry connected to diaper and to sensors in diaper to alert caregivers when diaper becomes wet or full; audio-visual wetness/bowel movement alert circuitry; algorithms to identify what sensor(s) discover and report it to the “Nurse Remote Wand” or to wireless/wired receiver links to computer database(s); a software-database system to allow staff to input data relating to treatment plans, diagnoses, notes, and to track editable patient data which is password protected; a database where sensor data on each patient is stored; a “Nurse Remote Wand” receives sensor data which is downloaded to a computer patient treatment and planning database; readouts to display sensor data.

### CROSS REFERENCE TO RELATED APPLICATIONS

This application is based on provisional application serial No. 60/464,420, filed on Mar. 22, 2003.

### STATEMENT REGARDING FEDERALLY SPONSORED RESEARCH OR DEVELOPMENT

Not Applicable

### DESCRIPTION OF ATTACHED APPENDIX

Not Applicable

### BACKGROUND OF THE INVENTION

This invention relates generally to the field of infant and adult sanitary hygiene and medical treatment and diagnostic and control products and more specifically to a machine or device for hygiene and care requirements providing a more hygienic diaper, sensor pad, and or sensing belt with alert, readout, transmission, paging, software and patient information database recording means for treating and caring for wetness, feces, and disease.

### FIELD OF THE INVENTION

The present invention relates generally to a very hygienic and highly advanced diaper that is proposed with alert means for wetness, and for controlling contact with urine and feces & disease and for eliminating diaper rashes and other diseases and for controlling diseases that are detectable by analyzing anal, penile, and vaginal secretions, including detection and analyses alerting and reporting functions for body temperature, urine, feces, and or other materials expelled in a diaper, by analyzing skin, the gasses, materials and or the liquids expelled or that which comes into contact with the diaper via sensors designed to detect, analyze and report body temperature, diseases, bacteria, viruses, parasites, abnormal conditions, drugs, and or other sensors or probes (such as temperature probes).

### BACKGROUND OF THE INVENTION

The invention is also designed to increase the comfort of infants, toddlers, children and older adults who may wear and use diapers and need them and is designed to improve sanitary conditions where weakened immune systems are characteristic in these age brackets where bacteria and viruses abound from urine and feces. The invention creates a more sanitary environment where caregivers will know immediately when urine and or feces have been expelled to correct the condition.

The invention provides means to communicate to caregivers that a child or an adult under care may have urinated or expelled matter into their diapers so the care giver may change them to create a more sanitary condition as immediately as possible to comfort the patient or child to promote a healthier condition, by applying medications, and to provide comfort and to relieve stress to the caregiver from crying or other disturbances that a child or a patient may be verbalizing.

Another problem that this invention solves is that the invention can be used as a diagnostic tool at home or in hospitals or in nursing homes, day care centers or in other facilities. The proposed invention allows the construction of different models, one for sensing and alerting the caregiver if the diaper has been filled with urine and or feces via a wetness sensor, and other models may be developed where sensors can be added to detect illness or other abnormalities where sensor(s) may detect blood in the urine, abnormal ketone levels, other impurities in the urine, and other dangerous conditions including to report body temperature or fever conditions with alerts thereof. A number of sensors can be used including liquid analysis sensors and gas-odor sensors that can be sandwiched in between the absorbent layers of the diaper material. Sensors can be placed in various positions throughout the diaper, either throughout the entire lining of the diaper, as a foil-type sensor, or other sensors, sandwiched between the absorbent layers 1 and 2 or within an absorbent layer if there is one layer or directly under the permeable layer or in contact with the wearer of the diaper if the sensor can be woven into the diaper in a comfortable and in a hygienic fashion.

Different sensors can have different uses, to sense wetness, and also to analyze vaginal or penile discharge and secretions for abnormalities, bacteria, viruses, and or disease. Sensor(s) may be disposable or re-useable in nature and the diaper may be designed in a fashion where the diaper may be opened in a fashion to insert either new sensor(s) in strategic area(s) for sensing. In diaper models where re-useable sensor(s) are used the sensor(s) may be designed to be sterilizable and be the type that can be used from one patient to another or to be used in multiple-use situations.

Where inexpensive sensor(s) are used, disposable sensor(s) can be built into the diapers, and they can be built-in to the diapers for throw-away use.

Specialized sensors are also introduced in the present invention to fully analyze the contents and the consistency of bowels or feces to report the health and condition of the patient also to monitor urine and penile and vaginal secretions and their condition and contents and to report the same and store information of the same, including the time of discharge in memory into logic circuits of the unit which can be uploaded to a nurse remote wand and later downloaded to computer and further analyzed and dumped to databases of patient information and studied through software and graphical information for doctors and caregivers and nursing staff to study to compare with medical treatment and drugs being administered.

### DESCRIPTION OF THE RELATED ART

It can be appreciated that diapers have been in use for years. Typically, diapers are comprised of diapers that are disposable or that are re-usable and that are launderable.

The main problem with conventional diapers is that they are not as hygienic and as sanitary as our proposed diaper designs are and that they are not designed to eliminate diaper rashes and other diseases as our diaper designs.

Another problem with conventional more hygienic sanitary diaper are that they do not provide means to alert and to communicate to caregivers that a child or an adult under care may have urinated or expelled matter into their diapers so the care giver may change them to create a more sanitary condition as immediately as possible to comfort the patient or child to promote a healthier condition, nor do they provide means to alert caregivers as to conditions of diaper wearer to treat and diagnose diseases, to detect impurities, blood, viruses, bacteria, or other abnormalities, for conditions including but not limited to abnormal ketone levels, sugar levels and or other dangerous conditions from vaginal, penile and anal secretions, nor do they assist caregivers in applying medications for treatment. Another problem with conventional more hygienic sanitary diaper are that they do not provide means to eliminate prolonged contact with urine or fecal matter and the bacteria and or the viruses that these contaminants have in infants, toddlers, children, and older adults who may have weak immune systems.

Furthermore, another problem with current diapers is that they are not currently used as a diagnostic tool at home or in hospitals or in nursing homes, day care centers or in other facilities.

The proposed invention allows the construction of different models, one for sensing and alerting the caregiver if the diaper has been filled with urine and or feces via a wetness sensor, and other models where sensors can be added to detect illness, or other abnormalities, where a sensor may detect blood in the urine, abnormal ketone levels, other impurities in the urine, and other dangerous conditions such as to report body temperature or fever conditions.

Sensors can be placed in various positions throughout the diaper, either throughout the entire lining of the diaper sandwiched between the absorbent layers 1 and 2 or within an absorbent layer if there is one layer or directly under the permeable layer or in contact with the wearer of the diaper if the sensor can be woven into the diaper in a comfortable and in a hygienic fashion. Different sensors can have different uses, to sense wetness, and also to analyze vaginal or penile discharge and secretions for abnormalities, bacteria, viruses, and or disease. A number of sensors can be used including liquid analysis sensors and gas-odor sensors that can be sandwiched in between the absorbent layers of the diaper material.

While these devices may be suitable for the particular purpose to which they address, they are not as suitable for eliminating diaper rashes and other diseases and for controlling diseases that may be detectable by analyzing urine and feces by analyzing the gasses and or the liquids expelled in the diaper. The invention is also designed to increase the comfort of infants, toddlers, children and older adults who may wear and use diapers and need them and is designed to improve sanitary conditions where weakened immune systems are characteristic in these age brackets where bacteria and viruses abound from urine and feces. The invention creates a more sanitary environment where caregivers will know immediately when urine and or feces have been expelled to correct the condition.

The invention provides means to communicate to caregivers that a child or an adult under care may have urinated or expelled matter into their diapers so the care giver may change them to create a more sanitary condition as immediately as possible to comfort the patient or child to promote a healthier condition and to provide comfort and to relieve stress to the caregiver from crying or other disturbances that a child or a patient may be verbalizing. Another problem that this invention solves is that the invention can be used as a diagnostic tool at home or in hospitals or in nursing homes, day care centers or in other facilities. The proposed invention allows the construction of different models, one for sensing and alerting the caregiver if the diaper has been filled with urine and or feces via a wetness sensor, and other models where sensors can be added to detect illness or other abnormalities where a sensor may detect blood in the urine, abnormal ketone levels, other impurities in the urine, and other dangerous conditions such as to report body temperature or fever conditions.

A number of sensors can be used including liquid analysis sensors and gas-odor sensors that can be sandwiched in between the absorbent layers of the diaper material. Sensors can be placed in various positions throughout the diaper, either throughout the entire lining of the diaper, as a foil-type sensor, or other sensors, sandwiched between the absorbent layers 1 and 2, or within multiple layers, or within an absorbent layer if there is one layer or directly under the permeable layer or in contact with the wearer of the diaper if the sensor can be woven into the diaper in a comfortable and in a hygienic fashion. Different sensors can have different uses, to sense wetness, and also to analyze vaginal or penile discharge and secretions for abnormalities, bacteria, viruses, and or disease.

The main problem with conventional diapers are that they are not as hygienic and as sanitary as our proposed diaper designs are because they are not designed to eliminate diaper rashes and other diseases. By alerting caregivers of a wetness condition when urination and or fecal expulsion occurs a caregiver may change a diaper immediately thus minimizing contact of these contaminants with the skin immediately thus eliminating diaper rash and other diseases associated with urination and fecal matter expulsion creating a significantly more sanitary condition and also providing greater comfort for the patient or the infant. Also, no conventional diaper systems are designed to alert caregivers of wetness or disease and other abnormalities, as our diaper designs do by transmitting sensor findings to a portable “nurse remote wand” with readout means to display sensor findings on individual patients for caregiver and medical staff convenience, and no diaper systems have the ability to display what the sensors have discovered along with concentration data to display the quantities, or the strength of each item discovered in the agents being detected by the sensors in the diaper systems herein described as our diaper systems do. Another problem with conventional diapers is that they do not provide means to communicate to caregivers that a child or an adult under care may have urinated or expelled matter into their diapers so the care giver may change them to create a more sanitary condition as immediately as possible to comfort the patient or child to promote a healthier condition and to apply medication or to cleanse the infant or patient, and or to apply lotions. Another problem with conventional diapers is that they do not provide a portable readout system apart from the diaper which can be problematic in hospital, day care or nursing home situations where there are multiple patients to be monitored.

Our diaper, sensor pad, and or sensor belt systems provide a “nurse remote wand” that can receive and display sensor data findings and give wetness and bowel movement alerts, along with sensor finding readouts on what the sensors have discovered and the concentrations of what was discovered from the diaper's “circuit card unit” transmitter through wired or wireless means so that caregivers can be alerted and so that diapers can be changed as soon as they are made wet or filled, and also to report medical and diagnostic medical, or disease conditions that the sensors have detected to the “nurse remote wand” or directly to computer databases in alternative versions of the invention.

Also, another problem is that they do not provide means to eliminate prolonged contact with urine or fecal matter and the bacteria and or the viruses that these contaminants have in infants, toddlers, children, and older adults who may have weak immune systems.

Furthermore, another problem with current diapers is that they are not currently used as a diagnostic tool at home or in hospitals or in nursing homes, day care centers or in other facilities. The proposed invention allows the construction of different models, one for sensing and alerting the caregiver if the diaper has been filled with urine and or feces via a wetness sensor, and other models where sensors can be added to detect illness, or other abnormalities, where a sensor may detect blood in the urine, abnormal ketone levels, other impurities in the urine, and other dangerous conditions such as to report body temperature or fever conditions.

Another problem with conventional diapers and related systems is that they are not designed to report body temperature or fever that may exist by the diaper wearer as our system(s) can.

Sensors can be placed in various positions throughout the diaper, either throughout the entire lining of the diaper sandwiched between the absorbent layers 1 and 2, or within multiple layers, or within an absorbent layer if there is one layer, or directly under the permeable layer, or in contact with the wearer of the diaper if the sensor can be woven into the diaper in a comfortable and in a hygienic fashion (see drawings).

Different sensors can have different uses, to sense wetness, and also to analyze vaginal, penile and or anal discharge and secretions for abnormalities, bacteria, viruses, and or disease. A number of sensors can be used including liquid analysis sensors and gas-odor sensors that can be sandwiched in between the absorbent layers of the diaper material.

In these respects, the hygienic diaper and or sensor pad with alert means for wetness, feces & disease according to the present invention substantially departs from the conventional concepts and designs of the prior art, and in so doing provides an apparatus primarily developed for the purpose of eliminating diaper rashes and other diseases and for diagnosing, and treating and controlling diseases, and for applying medications for such diseases and abnormalities that may be detectable by analyzing urine and feces and secretions by analyzing the gasses and or the liquids expelled in the diaper from penile, vaginal and anal secretions.

The invention is also designed to increase the comfort of infants, toddlers, children and older adults who may wear and use diapers and need them and is designed to improve sanitary conditions where weakened immune systems are characteristic in these age brackets where bacteria and viruses abound from urine and feces. The invention creates a more sanitary environment where caregivers will know immediately when urine and or feces have been expelled to correct the condition.

The invention provides means to communicate to caregivers that a child or an adult under care may have urinated or expelled matter into their diapers so the care giver may change them to create a more sanitary condition as immediately as possible to comfort the patient or child to promote a healthier condition and to provide comfort and to relieve stress to the caregiver from crying or other disturbances that a child may be verbalizing.

Another problem that this invention solves is that the invention can be used as a diagnostic tool at home or in hospitals or in nursing homes, day care centers or in other facilities. The proposed invention allows the construction of different models, one for sensing and alerting the caregiver if the diaper has been filled with urine and or feces via a wetness sensor, and other models where sensors can be added to detect illness or other abnormalities where a sensor may detect blood in the urine, abnormal ketone levels, other impurities in the urine, and other dangerous conditions such as to report body temperature or fever conditions.

A number of sensors can be used including liquid analysis sensors and gas-odor sensors that can be sandwiched in between the absorbent layers of the diaper material. Sensors can be placed in various positions throughout the diaper, either throughout the entire lining of the diaper, as a foil-type sensor, or other sensors, sandwiched between the absorbent layers 1 and 2, within multiple layers in multiple layer diapers, or within an absorbent layer if there is one layer, or directly under the permeable layer or in contact with the wearer of the diaper if the sensor can be woven into the diaper in a comfortable and in a hygienic fashion.

Different sensors can have different uses, to sense wetness, and also to analyze anal, vaginal or penile discharge and secretions for abnormalities, bacteria, viruses, and or disease found in medicine.

### SUMMARY OF THE INVENTION

In view of the foregoing disadvantages inherent in the known types of diapers now present in the prior art, the present invention provides a new hygienic diaper and or sensor pad with alert means for wetness, feces & disease whose construction wherein the same can be utilized for eliminating diaper rashes and other diseases and for controlling diseases that may be detectable by analyzing urine and feces and secretions by analyzing the gasses and or the liquids expelled into the diaper. The invention is also designed to increase the comfort of infants, toddlers, children, adults, and older adults who may wear and use diapers and need them and is designed to improve sanitary conditions where weakened immune systems are characteristic in these age brackets where bacteria and viruses abound from urine and feces.

The invention creates a more sanitary environment where caregivers will know immediately when urine and or feces have been expelled to correct the condition.

The invention provides means to communicate to caregivers that a child or an adult under care may have urinated or expelled matter into their diapers so the care giver may change them to create a more sanitary condition as immediately as possible to comfort the patient or child to promote a healthier condition and to provide comfort and to relieve stress to the caregiver from crying or other disturbances that a child may be verbalizing. Another problem that this invention solves is that the invention can be used as a diagnostic tool at home or in hospitals or in nursing homes, day care centers or in other facilities.

The proposed invention allows the construction of different models, one for sensing and alerting the caregiver if the diaper has been filled with secretions, urine and or feces via a wetness sensor, and other models where sensors can be added to detect illness or other abnormalities where a sensor may detect blood in the urine, abnormal ketone levels, other impurities in the urine, and other dangerous conditions such as to report body temperature or fever conditions. A number of sensors can be used including liquid analysis sensors and gas-odor sensors that can be sandwiched in between the absorbent layers of the diaper material. Sensors can be placed in various positions throughout the diaper, either throughout the entire lining of the diaper, as a foil-type sensor, or other sensors, sandwiched between the absorbent layers 1 and 2 or within an absorbent layer if there is one layer or directly under the permeable layer or in contact with the wearer of the diaper if the sensor can be woven into the diaper in a comfortable and in a hygienic fashion.

Different sensors can have different uses, to sense wetness, and also to analyze anal, vaginal or penile discharge and secretions for abnormalities, bacteria, viruses, and or disease found in medicine.

The general purpose of the present invention, which will be described subsequently in greater detail, is to provide a new hygienic diaper and or sensor pad with alert means for wetness, feces & disease that has many of the advantages of the more hygienic sanitary diaper mentioned heretofore and many novel features that result in a new hygienic diaper and or sensor pad with alert means for wetness, feces & disease which is not anticipated, rendered obvious, suggested, or even implied by any of the prior art more hygienic sanitary diaper, either alone or in any combination thereof.

To attain this, the present invention generally comprises a diaper preferably made out of disposable materials, but not limited to disposable materials, with absorbent layers with a wetness sensor and other sensors sandwiched between the layers of the diaper in the deluxe model. In the various models, there will be sensors in the lining of the diaper, composed of a foil lining, or other sensors, sandwiched between the absorbent layers of the diaper attached to a “circuit card unit” to sense wetness, odor, disease, bacteria, and or bacteria levels, blood, viruses, or other abnormalities, etc. The “circuit card unit” is attached to the diaper and is attached to the sensors through “electrode snaps” on the waist line of the diaper will alert the caregiver(s).

The circuit card unit, in the deluxe models for hospitals will have a transmission circuit that will transmit an ID for the diaper and that will alert the caregiver or nurse, on their remote wands, that a diaper is wet or that a disease or other abnormality has been sensed in deluxe models.

Of course, various models can be marketed, one may be wetness only to detect whether urination has occurred, and or usually followed with feces expulsion, or other diaper models can be developed and marketed targeted to diagnose and sense for different conditions and diseases each to be manufactured with different sensors.

The diapers can also be used in facilities as a method for stool collection. The Outer Shell Of The Disposable Diaper (Is Made Of A Plastic-Type Material). Absorbent Layer(s) Of Diaper. (The invention herein implies that it is understood that a diaper may be constructed with just 1 absorbent layer to a maximum of “n” layers, and that sensors may be strategically embedded where appropriate to provide the benefit intended). Foil Wetness Lining Sensor Sandwiched Between Absorbent Layers Of Diaper. (The invention herein implies that it is understood that a diaper may be constructed with just 1 absorbent layer to a maximum of “n” layers, and that sensors may be strategically embedded where appropriate to provide the benefit intended). Electrode Snaps Communicate From Sensors To Small Circuit Card Unit That Snaps On To Diaper. Circuit Card Unit Senses Wetness, Disease, And Abnormalities, And Connects To Sensors.

Other Sensors Designed To Detect Disease And Abnormalities Sandwiched Between Absorbent Layers Of The Diaper. (The invention herein implies that it is understood that a diaper may be constructed with just 1 absorbent layer to a maximum of “n” layers, and that sensors may be strategically embedded where appropriate to provide the benefit intended).

The Circuit Card Unit Can Operate Via A Battery Or Via An AC Adapter For Hospitalized Patients Confined To Hospital Beds. The Permeable Lining Between The Infant Or Adult And “Absorbent Layer #1”, Designed To Allow Absorption Into The Absorbent Layers Of The Diapers. Nurse Remote Wand Is Used For Monitoring Deluxe Versions Of Diapers I Hospital Or Special Care Facilities Where There Are Multiple Patients And Sanitary Conditions Are To Be Observed.

There has thus been outlined, rather broadly, the more important features of the invention in order that the detailed description thereof may be better understood, and in order that the present contribution to the art may be better appreciated. There are additional features of the invention that will be described hereinafter.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

The primary object of the invention is to provide a very hygienic and highly advanced diaper system that will alert caregivers for wetness, and for controlling and minimizing contact with urine, feces, and disease and for eliminating diaper rashes and other diseases and for controlling diseases that are detectable by analyzing anal, penile, and vaginal or skin secretions, including analyzing urine and feces by analyzing the gasses and or the liquids expelled in the diaper.

Another object of the invention is to provide a diaper system designed to care for those that may have weakened immune systems or other illnesses that may benefit from added hygiene care.

Another object of the invention is to assist caregivers to provide a more sanitary condition to toddlers, or adults under their care that wear diapers.

A further object of the invention is to provide automated electronic means for caregiving and the treatment of wetness and disease associated with urine and bowel movements.

Yet another object of the invention is to provide automated electronic means for nursing and doctors to track conditions of patients having urinary or bowel related disorders.

Still yet another object of the invention is to provide automated data collection and medical recording means for nursing and doctors for treating illness and tracking individual patient treatment plans and patient progress for disease and pathogens sensed through sensors in diapers.

Another object of the invention is to alert caregivers when diapers have been made wet or when diapers have received bowel movements to provide a more hygienic environment.

Another object of the invention is to increase the comfort of adults, infants or children under caregiver control through automatic alert means when diaper wetness or bowel movements occur.

A further object of the invention is to reduce the stress of caregivers by comforting diaper wearers when wetness or bowel movements occur.

Yet another object of the invention is to reduce disease, skin conditions, and rashes that are associated with wet diapers or diapers that have bowel movements in them and prolonged contact with skin by alerting caregivers to change diapers.

Still yet another object of the invention is to provide nursing staff and doctors with a means to monitor groups of patients with a “Nurse Remote Wand” that identifies patients that wet their diaper or that have bowel movements so that diapers can be changed immediately.

Another object of the invention is to provide a diaper with interchangeable sensors to sense bacteria, viruses, or drugs taken by the patient that can be analyzed and reported by the sensor(s).

Another object of the invention is to provide diaper(s) that are reusable or disposable either with inexpensive throw away sensor(s) or with specific sensors that can be sanitized or sterilized for re-use.

A further object of the invention is to provide a diaper that can be used as a diagnostic tool for medical purposes through sensor and data recording means.

Yet another object of the invention is to provide a diaper with data and system features, alert and reporting functions that will relieve stress for caregivers and assist in the treatment of patients for hospitals, nursing homes or day care centers, with different models for home use.

Still yet another object of the invention is to provide a diaper with sensor means to sense detect and analyze ketone levels, sugar levels, blood, body fluids, liquids, or gasses given off by the wearer in addition to bacteria, toxins, viruses, drugs or other items or impurities that may be sensed through sensor means.

Another object of the invention is to provide a diaper system with circuitry that takes sensor information and transmits an alert to caregivers when wetness or bowel movements occur and medical findings data through a transmission circuit with a patient ID to a “Nurse Remote Wand” for monitoring purposes.

Another object of the invention is to provide a software interface and database system that will allow nursing staff and doctors to look up data on sensor information collected on each patient and to track drug treatment plans, diagnoses, patient records, notes, and patient progress that may be edited and printed in report form as needed which is accessible through a multi-level password system to have multiple access rights for doctors and nursing staff.

A further object of the invention is to alert the caregivers that wetness has occurred through visual and or audible means either through the “circuit card unit” on the diaper itself, or from the “Nurse Remote Wand” once a transmission is received from the “circuit card unit”.

Yet another object of the invention is to provide a diaper that will clock and keep track of when the patient or diaper wearer wets the diaper or has a bowel movement electronically and update his or her database accordingly for treatment and patient progress.

Still yet another object of the invention is to provide a diaper system with software functions that will allow tracking of sensor detection data found by sensors, patient data, treatment plans, patient progress, response to treatment, condition of urine, including such data such as “weight of full diaper”, “consistency of bowel”, “color of bowel”, etc., for patient analyses of “patient progress and condition”; patient database will also have user defined fields.

Another object of the invention is to have sensor signals adjusted through circuitry in the “circuit card unit” to adjust their sensitivity to be more or less sensitive through adjustment means on the “circuit card unit” by applying amplification or resistance to the signals as desired through control means that a caregiver can adjust through selective control means or buttons on the “circuit control card”.

Another object of the invention is to provide a diaper with one or more sensor(s) to sense one or more variables, such as disease, drugs, antibodies, proteins, wetness, bowels, urine, ketone levels, sugar levels, parasites, bacteria, viruses, sebum, secretions, body fluids, gasses, liquids, materials, toxins, or any item that might be of interest that sensor(s) may be designed to sense.

Another object of the invention is to provide a “circuit card unit” to be placed on the diaper to connect to the sensor(s) of the diaper that will be water-resistant or water-proof connecting and functioning with the sensors so that it will not short circuit and that it will continue to operate even when a diaper becomes wet.

Another object of the invention is to provide a “circuit card unit” to be placed on the diaper that will be small and flexible for the comfort and safety of the wearer.

Another object of the invention is to provide a diaper that will provide means to strategically attach and position anal, vaginal, and penile temperature thermometers and or disease sensor(s) or probes to the diaper so they will be easily inserted into the anal, vaginal and penile canals through flexibly fitting and positional sensors to a patient.

Another object of the invention is to provide a strap-on diaper with built-in attached strategically placed sensors designed for anal, vaginal or penile canal readings whereby a patient is prevented from expelling the sensor(s) from said canals through voluntary or through involuntary muscular contractions because the diaper holds the sensor(s) in their inserted position(s) as the diaper is strapped on the patient or the wearer.

Another object of the invention is to provide a “circuit card unit” and sensors that will track the frequency and time of gas discharge and report the same via sensor(s).

Another object of the invention is to provide a “nurse remote wand” with an optional “magnetic card reader” built into it for reading patient identification cards containing medical information that can be stored into the “nurse remote wand” and also later downloaded to a computer to a patient database along with sensor information picked up from diaper sensor(s).

Another object of the invention is to provide a “nurse remote wand” with an optional “magnetic card reader” jack built into it for attaching a separate “magnetic card reader” to the “nurse remote wand” for reading patient identification cards containing medical information that can be stored into the “nurse remote wand” and also later downloaded to a computer to a patient database along with sensor information picked up from diaper sensor(s).

Another object of the invention is to provide a diaper with a detachable sanitary and water-tight and sealable “tear-off stool-urine-materials collection pouch” in addition to the afore-mentioned alert diaper disease control system that will allow “stool-urine-materials” collection as part of the treatment process, with a section on it that a lab technician may record the patient's name and or other information or notes.

A primary object of the present invention is to provide very hygienic and highly advanced diaper that is proposed to alert caregivers for wetness, and for controlling and minimizing contact with urine and feces & disease and for eliminating diaper rashes and other diseases and for controlling diseases that are detectable by analyzing anal, penile, and vaginal secretions, including analyzing urine and feces by analyzing the gasses and or the liquids expelled in the diaper that will overcome the shortcomings of the prior art devices.

An object of the present invention is to provide a very hygienic and highly advanced diaper that is proposed to alert caregivers for wetness, and for controlling and minimizing contact with urine and feces & disease and for eliminating diaper rashes and other diseases and for controlling diseases that are detectable by analyzing anal, penile, and vaginal secretions, including analyzing urine and feces by analyzing the gasses and or the liquids expelled in the diaper.

The invention is also designed to increase the comfort of infants, toddlers, children, adults, and older adults who may wear and use diapers and need them and is designed to improve sanitary conditions where weakened immune systems are characteristic in these age brackets where bacteria and viruses abound from secretions, urine and feces. The invention creates a more sanitary environment where caregivers will know immediately when urine and or feces have been expelled to correct the condition. The invention provides means to communicate to caregivers that a child or an adult under care may have urinated or expelled matter into their diapers so the care giver may change them to create a more sanitary condition as immediately as possible to comfort the patient or child to promote a healthier condition and to provide comfort and to relieve stress to the caregiver from crying or other disturbances that a child or patient may be verbalizing. Another problem that this invention solves is that the invention can be used as a diagnostic tool at home or in hospitals or in nursing homes, day care centers or in other facilities. The proposed invention allows the construction of different models, one for sensing and alerting the caregiver if the diaper has been filled with urine and or feces via a wetness sensor, and other models where sensors can be added to detect illness or other abnormalities where a sensor may detect blood in the urine, abnormal ketone levels, other impurities in the urine, or feces, or secretions, and other dangerous conditions such as to report body temperature or fever conditions.

A number of sensors can be used including liquid analysis sensors and gas-odor sensors that can be sandwiched in between the absorbent layers of the diaper material. Sensors can be placed in various positions throughout the diaper, either throughout the entire lining of the diaper, as a foil-type sensor, or other sensors, sandwiched between the absorbent layers 1 and 2, or within multiple layers, or within an absorbent layer if there is one layer or directly under the permeable layer or in contact with the wearer of the diaper if the sensor can be woven into the diaper in a comfortable and in a hygienic fashion. Different sensors can have different uses, to sense wetness, and also to analyze vaginal or penile discharge and secretions for abnormalities, bacteria, viruses, and or disease.

Another object is to provide an advanced hygienic diaper and or sensor pad with alert means for sensing wetness, feces & disease that will sense wetness once secretions, urine or feces are expelled into the diaper and alert the caregiver through visual and or audible means.

Another object of the invention is to provide a diaper system with a “nurse remote wand” that can receive and display sensor data findings and give wetness and bowel movement alerts, along with sensor finding readouts on what the sensors have discovered and the concentrations of what was discovered from the diaper's “circuit card unit” transmitter through wired or wireless means so that caregivers can be alerted and so that diapers can be changed as soon as they are made wet or filled, and also to report medical and diagnostic medical, or disease conditions that the sensors have detected to the “nurse remote wand” or directly to computer databases in alternative versions of the invention.

Another object is to provide a very hygienic and highly advanced diaper that is proposed to alert caregivers for wetness, and for controlling and minimizing contact with urine and feces & disease and for eliminating diaper rashes and other diseases and for controlling diseases that are detectable by analyzing anal, penile, and vaginal secretions, including analyzing urine and feces by analyzing the gasses and or the liquids expelled in the diaper that will have a sensor in the lining of the diaper, composed of a foil lining, and or other sensor, sandwiched between the absorbent layers of the diaper attached to a circuit to sense wetness or odor or of disease or bacteria levels, or other abnormalities, that will alert the caregiver(s). The circuit, in the deluxe models for hospitals will have a transmission circuit that will transmit an ID for the diaper and that will alert the caregiver or nurse, on their remote wands, that a diaper is wet or that a disease or other abnormality has been sensed in deluxe models.

Sensors can be placed in various positions throughout the diaper, either throughout the entire lining of the diaper sandwiched between the absorbent layers 1 and 2, within multiple layers, or within an absorbent layer if there is one layer or directly under the permeable layer or in contact with the wearer of the diaper if the sensor can be woven into the diaper in a comfortable and in a hygienic fashion. Different sensors can have different uses, to sense wetness, and also to analyze anal, vaginal or penile discharge and secretions for abnormalities, bacteria, viruses, and or disease. Another object of my invention is to provide a device that will allow a diaper to be constructed that will allow one to open the diaper up in a fashion to insert and use expensive new one-time use or re-useable sensors that may be more expensive, and which would interface and communicate with the “circuit card unit”, and which may be sterilized for use from one patient to another or from one diaper to another.

This may provide for different models of diapers in addition to other models of diapers that will have sensors that are built-in and are inexpensive and that are disposable in nature.

Another object is to provide a very hygienic and highly advanced diaper that is proposed to alert caregivers for wetness, and for controlling and minimizing contact with urine and feces & disease and for eliminating diaper rashes and other diseases and for controlling diseases that are detectable by analyzing anal, penile, and vaginal secretions, including analyzing urine and feces by analyzing the gasses and or the liquids expelled in the diaper that has sensing capabilities that will alert the caregiver(s) if wetness has occurred or if dangerous levels of certain substances are found in a child's or an adult's urine, secretions, or feces, such as blood, abnormal ketone levels, abnormal sugar levels, bacteria, viruses, other abnormalities, or if other abnormal items are found in medicine such as problems with body temperature or fever that a particular sensor placed in the diaper can analyze are found electronically such as to report low or high body temperature or fever conditions, or to report normal body temperature, or the exact body temperature through electronic temperature sensors or probes in our diaper system.

Another object is to provide a very hygienic and highly advanced diaper that is proposed to alert caregivers for wetness, and for controlling and minimizing contact with urine and feces & disease and for eliminating diaper rashes and other diseases and for controlling diseases that are detectable by analyzing anal, penile, and vaginal secretions, including analyzing urine and feces by analyzing the gasses and or the liquids expelled in the diaper that has sensing capabilities that will alert the caregiver(s) if wetness has occurred or if dangerous levels of certain substances are found in a child's or an adult's urine, secretions, or feces, such as blood, abnormal ketone levels, abnormal sugar levels, bacteria, viruses, other abnormalities, or if other abnormal items are found in medicine.

The particular sensor(s) placed in the diaper can analyze abnormalities through diapers having chemical sensors that change color or that provide information without the use of electronic components or without circuit cards where an indicator changes color or where an indicator provides information is built into the diaper as a diagnostic tool.

Another object is to provide a very hygienic and highly advanced diaper that is proposed to alert caregivers for wetness, and for controlling and minimizing contact with urine and feces & disease and for eliminating diaper rashes and other diseases and for controlling diseases that are detectable by analyzing anal, penile, and vaginal secretions, including analyzing urine and feces by analyzing the gasses and or the liquids expelled in the diaper that has sensing capabilities that will alert the caregiver(s) if wetness has occurred or if dangerous levels of certain substances are found in a child's or an adult's urine, secretions, or feces, such as blood, abnormal ketone levels, abnormal sugar levels, bacteria, viruses, other abnormalities, or other abnormal items are found in medicine. The particular sensor(s) placed in the diaper can analyze abnormalities through diapers containing both electronic and chemical sensors that change color and or that provide information. This hybrid diaper will use both electronic components or circuit cards where an chemical sensor with an indicator that changes color or that provides information can be used in tandem with electronic sensors will be built into the diaper as a diagnostic tool combined with electronic sensors to form diapers with special sensing functionalities for specialized diagnostic abilities.

Another object is to provide means to strategically attach and position removable and or adjustable anal, vaginal, and penile temperature thermometers and or disease sensor(s) or probes to the diaper so they will be easily inserted into the anal, vaginal and penile canals through flexibly fitting and positional sensors to a patient in fixed and or adjustable strategic position(s) with flexibility having at least three comfortable flexible sensor(s): an anal sensor of “x” length and “y” thickness, a vaginal sensor of “x” length and “y” thickness, and or a penile sensor of “x” length and “y” thickness [where “x” and “y” can be the same and or different number(s) for any and all sensor(s)], to be positioned and inserted into anal, vaginal, and penile canals respectively, where upon the wearer being fit with the diaper with sensors in place wearing the diaper prevents the sensors from being expelled from voluntary or involuntary muscular contraction(s) by virtue of holding the sensors securely into place after insertion by the patient wearing the diaper.

Another object of the invention is to have anal, vaginal and or penile sensors that can be inserted in anal, vaginal and or penile canals whereby said sensors are removable and adjustably positional and whereby the sensors can slide bi-directionally in a either a continuous groove or in separate grooves whereby the sensors may slide bi-directionally within said groove(s) within the absorbent layer(s) of the diaper to comfortably adjust and correctly position and insert the sensor(s) into the anal, vaginal, and penile canals respectively in a comfortably fitting manner for different size requirements of specific individuals and whereby the heads of the sensor(s) are large enough and tight-fitting enough within said groove(s) that once adjusted they remain in their adjusted position(s) and whereby the sensor(s) remain supported in their upright and or in their functional flexible position(s) for a comfortable fit or insertion into their anal, vaginal or penile canals for temperature, disease or other detection interest(s); and where the anal, vaginal, and penile sensor probes may be plugged into or removed from a socket or wire connection within the groove(s), said sockets having electrodes or wires connecting to electrode or wire connections to other sensors within the sensor layer or directly to the electrode or wire connections leading to connection(s) or “electrode snaps” on the outer portion of the diaper where the “circuit card unit(s)” may be attached to said electrodes to communicate with said sensor(s); and whereby in the case of the penile sensor, the penile sensor probe may have its distance extended from the diaper by utilizing a probe having a wired connection of “x” length connecting to the diaper at a socket or a wire contact location where the penile sensor can be attached to the diaper and where the penile sensor may then be placed manually into the penile canal to accommodate the easy placement of the penile sensor into the penile canal.

Another object is to provide a very hygienic and highly advanced diaper that is proposed to alert caregivers for wetness, and for controlling and minimizing contact with urine and feces & disease and for eliminating diaper rashes and other diseases and for controlling diseases that are detectable by analyzing anal, penile, and vaginal secretions, including analyzing urine and feces by analyzing the gasses and or the liquids expelled in the diaper that will allow a diaper to be used as a diagnostic tool at home or in hospitals or in nursing homes, day care centers or in other facilities. The proposed invention allows the construction of different models, one for sensing and alerting the caregiver if the diaper has been filled with urine and or feces via a wetness sensor, and other models where sensors can be added to detect illness where a sensor may detect blood in the urine, abnormal ketone levels, other impurities in the urine, and other dangerous conditions. A number of sensors can be used including liquid analysis sensors and gas-odor sensors that can be sandwiched in between the absorbent layers of the diaper material.

Another object is to provide a very hygienic and highly advanced diaper that is proposed to alert caregivers for wetness, and for controlling and minimizing contact with urine and feces & disease and for eliminating diaper rashes and other diseases and for controlling diseases that are detectable by analyzing anal, penile, and vaginal secretions, including analyzing urine and feces by analyzing the gasses and or the liquids expelled in the diaper that will allow a home diaper to be constructed whereby once wetness is sensed by the sensor a flashing LED on a small circuit snapped onto the diaper will blink and or an audible beep will be heard until defeated by pressing a button 3 times or by some other defeat mechanism.

Another object of my invention is to provide a device that will allow a home diaper to have sensors as described herein to alert the caregiver that disease, bacteria, viruses, impurities, blood, and or other abnormalities exist from vaginal or penile or anal secretions. Furthermore, sensors will be able to sense ketone levels, sugar levels and other items of interest to the medical community. Many different models of diapers can be made with specialized sensors for different uses.

Another object of the invention is to provide specialized sensors within the diaper that can determine the consistency of the bowels, or feces, and determine how soft or hard the bowels or feces are and discriminate between normal feces and abnormal feces, (also diseased or not) and also if diarrhea is present, and further to determine the amount of water or other contents in the feces, the types of microbes and or viruses in the feces or urine or other secretions and report the condition and store the information, along with percentages of contents found or elements found, along with time of discharge into memory which is later downloaded to the nurse remote wand or directly to a computer through a wireless port or to a direct connection wired to the diaper's sensor whose information is downloaded into patient databases and graphs for treatment history interfacing for medical and nursing teams and report generation and patient monitoring.

Another object of the invention is to provide specialized sensors within the diaper that can analyze the contents of feces and diarrhea, urine, penile, and vaginal secretions and report the condition and store the information, along with time of discharge into memory which can later be downloaded to the nurse remote wand or directly to a computer through a wireless port or to a direct connection wired to the diaper's sensor whose information is downloaded into patient databases and graphs for treatment history interfacing for medical and nursing teams and report generation and patient monitoring.

Another object is to provide a very hygienic and highly advanced diaper that is proposed to alert caregivers for wetness, and for controlling and minimizing contact with urine and feces & disease and for eliminating diaper rashes and other diseases and for controlling diseases that are detectable by analyzing anal, penile, and vaginal secretions, including analyzing urine and feces by analyzing the gasses and or the liquids expelled in the diaper that will allow diapers to be coded for hospitals with transmission capabilities to nurse remote wands to be constructed whereby once wetness is sensed by the sensor a flashing LED on a small circuit snapped onto the diaper will blink and or an audible beep will be heard along with the diaper's ID and a flashing LED on the nurse's remote wand will be found until the diaper signal is defeated by pressing a button 3 times on the patient's diaper circuit card or by some other defeat mechanism.

Another object is to provide a very hygienic and highly advanced diaper that is proposed to alert caregivers for wetness, and for controlling and minimizing contact with urine and feces & disease and for eliminating diaper rashes and other diseases and for controlling diseases that are detectable by analyzing anal, penile, and vaginal secretions, including analyzing urine and feces by analyzing the gasses and or the liquids expelled in the diaper that will allow a diaper to be constructed that will alert caregivers that disease or abnormalities exist in the urine or in the feces. The diaper will provide data and display such data to caregivers upon pressing a button from specialized sensors after gathering such data if diseases or abnormalities are sensed from the liquids and or the gasses that are given off from the urine and or the feces that are expelled in the diaper.

Furthermore, another object of this invention is to provide a device that will allow diapers to be coded for hospitals with transmission capabilities to nurse remote wands to be constructed whereby once disease or other abnormalities or wetness are sensed by the sensor a flashing LED on a small circuit snapped onto the diaper will blink and or an audible beep will be heard along with the diaper's ID and a flashing LED on the nurse's remote wand will be found until the diaper signal is defeated by pressing a button 3 times on the patient's diaper circuit card and then by depressing the “cancel alert button” on the “nurse remote wand” within one minute of depressing the circuit card three times while the patient's diaper ID number is on the “nurse remote wand” or by some other defeat mechanism.

Another object is to provide a very hygienic and highly advanced diaper that is proposed to alert caregivers for wetness, and for controlling and minimizing contact with urine and feces & disease and for eliminating diaper rashes and other diseases and for controlling diseases that are detectable by analyzing anal, penile, and vaginal secretions, including analyzing urine and feces by analyzing the gasses and or the liquids expelled in the diaper that will allow a diaper to be constructed that will alert caregivers that disease or other abnormalities exist in the urine, in secretions or in the feces through circuit and sensor means. It is contemplated that the circuit card unit will have the ability to analyze sensor information and display the appropriate warning(s) as to what it has sensed on an LCD readout and to provide an audible and a bright LED visual alert. In addition, it will give these alerts to a remote “nurse remote wand” through radio transmission means. All circuit means, including diaper ID information for multiple-patient monitoring will be transmitted to the “nurse remote wands” in the deluxe models. Input means on the “circuit control units” will have the ability to add diaper ID information for each diaper. In addition, the circuit card units will have memory to store the diaper ID information and the appropriate buttons to scroll through alert warnings and to cancel alerts and to turn the unit on or off and to clear its memory.

In addition the circuit card unit will have all the integrated circuits; wiring; a battery compartment; an AC adapter connection; a speaker for audible alerts; a Light Emitting Diode for visual alerts; a Liquid Crystal Display for wetness alerts where the words: “DIAPER WET” or another warning can be displayed when wetness is sensed; the default display on the Liquid Crystal Display would always read: “Diaper Dry”; the same Liquid Crystal Display can be used for displaying diseases or codes for such diseases and for abnormalities that are sensed from the secretions and or the gasses and the liquids that are expelled into the diaper from anal, penile, and vaginal secretions; connections and or terminal snap-fit connections to the electrode snaps and or separately to electronic sensors; buttons for programming the diaper ID on deluxe models; an antennae for transmitting the diaper ID to “nurse remote wands” on deluxe models; scrolling features to scroll through different warnings displayed on the unit (these warnings can range from wetness to a range of diseases or abnormalities sensed from the sensors utilized); a cancel button to cancel the alert; a power button to turn the circuit card unit on or off and to clear the circuit card unit's memory from the warnings and the alerts.

The circuit card unit will have all the electrical components required for the intended for the operations as herein described within this document in all its various models and can be constructed differently in different models having more and or less features to accommodate more or less functionalities with variations as desired depending on the sensors utilized and the functionalities desired.

It is contemplated that the home units will not require transmission means to be built into the circuit card unit where transmission will be sent to a “nurse remote wand” and many of the features on the “deluxe circuit card unit” will not be required for home units. Features not required on home units would be those where programmability for Diaper IDs would be available. The home unit “circuit card unit” shows the non-deluxe version as depicted in the drawings.

Another object is to provide a very hygienic and highly advanced diaper that is proposed to alert caregivers for wetness, and for controlling and minimizing contact with urine and feces & disease and for eliminating diaper rashes and other diseases and for controlling diseases that are detectable by analyzing anal, penile, and vaginal secretions, including analyzing urine and feces by analyzing the gasses and or the liquids expelled in the diaper that will allow a diaper to be constructed that will alert caregivers that disease and or other abnormalities that exist in the urine, in secretions or in the feces through circuit and sensor means and that will allow the clocking of the actual time when wetness or secretions are found by sensors or when anal, penile or vaginal secretions are expelled into the diaper and have been sensed by the circuit card unit. It is obvious by this functionality that a clock circuit will be included in the circuit card unit that keeps track of the time for this feature that will store the actual time of such expulsion(s) into the memory of the circuit card unit of the diaper and that a caregiver can scroll on the “circuit card unit” through the messages on the “LCD” display to check for the time of expulsion into the diaper.

In the deluxe version of the “circuit card units” for the hospital units, the caregiver can see this information on the “nurse remote wand” as well because the “actual time” will be transmitted immediately once wetness, secretion, disease identification, abnormality identification, and or expulsion occurs, and the “circuit card unit” will also display the same information that is attached to the diaper of the patient. On home units no “nurse remote wands” are used, but caregiver(s) can scroll through messages on the LCD to view the actual time of wetness, secretion, disease identification, abnormality identification, and or expulsion(s) as home units still have the same functionalities except for the “nurse remote wand” capability.

Another object is to provide a very hygienic and highly advanced diaper that is proposed to alert caregivers for wetness, and for controlling and minimizing contact with urine and feces & disease and for eliminating diaper rashes and other diseases and for controlling diseases that are detectable by analyzing anal, penile, and vaginal secretions, including analyzing urine and feces by analyzing the gasses and or the liquids expelled in the diaper that will allow a diaper to be constructed that will alert caregivers that disease and or other abnormalities that exist in the urine, in secretions or in the feces through circuit and sensor means and that will allow the dumping of patient data into computer files, databases, and for hard copy computer printouts on paper for the deluxe hospital models using the “nurse remote wands”. The “nurse remote wands” can be connected to a computer.

The patient data can be transferred to a computer database and printouts can be printed on all the patients for progress reports that will give caregiver(s) and doctors information on the actual time of wetness, secretion, disease identification, abnormality identification, and or expulsion(s) and it will identify the condition(s) sensed by the sensor(s) and provide that information to the caregiver(s) and the doctor(s) in report form. The system will also come with software that will enable doctors(s), nurses and caregiver(s) to weigh a clean diaper and weigh a full diaper after expulsion(s) and or secretion(s) have been expelled or secreted into the diaper so that information can also be inputted into the record for each patient. The Software provided will work automatically to combine information with a patient's Diaper ID number or identification that was programmed by caregivers and update records as time goes on with each diaper reset and cancellation when a new clean diaper is put on a patient. Graphing functionalities for the actual time of wetness, secretion, disease identification, abnormality identification, and or expulsion(s) into a diaper are built into the software as well as graphing functionalities for keeping track of the weight of a full diaper once matter has been expelled into the diaper, whether it is anal, penile, vaginal secretions or expulsions. Other features of the software will allow one to record the color of anal, penile, and vaginal secretion(s) and expulsion(s), their consistency, whether the patient had loose stools or diarrhea, and how the patient is responding to medication and to track progress. There is also a section where one can record notes for each patient in the record for each patient for each diaper change to track progress.

Another object of the present invention is to provide an alternate embodiment of the present invention by providing all the functionality of the present invention to a sensing pad (as opposed to a diaper) having “X” length and “Y” width” having an absorbent layer embedded with one or more sensor(s) that can be applied to a wound or to contaminated areas, or to spills, for sensing detection and reporting application applications the same way the diaper works to be connected through “n” number of electrode snaps in communication with the “circuit card unit” through “n” number of electrodes or wires, having optional adhesive attachment means on its edges to attach the pad to the skin or to an area for sensing, analyses, and reporting functions.

Yet another object of the present invention is to have the functionality to be able to code each sensor to pinpoint what area they are sensing or to identify the sensor(s) so when they report their data they also report their location, their sensor ID, and or their area they are sensing to help assist data gathering and information analyses needs.

Another object in developing various proposed models of the diaper herein is to provide a very hygienic and highly advanced diaper that is proposed to alert caregivers for wetness, and for controlling and minimizing contact with urine and feces & disease and for eliminating diaper rashes and other diseases and for controlling diseases that are detectable by analyzing anal, penile, and vaginal secretions, including analyzing urine and feces by analyzing the gasses and or the liquids expelled in the diaper that will allow a diaper to be constructed that will alert caregivers that disease and or other abnormalities that exist in the urine, in secretions or in the feces by constructing a diaper that will have medication(s), salves, ointments, powder(s), aloe, healing agents, or other lotions chemically added to the diaper in the absorbent layers or to other layer(s) of the diaper that come into contact with the wearer of the diaper for the treatment of diseases, abnormalities and or condition(s) associated with diaper rashes and or other diseases and condition(s) and abnormalities that can be treated by the wearing of a diaper through contact means or through transdermal transmission from the diaper to the skin.

Another object of the invention is to provide a diaper with a built-in sanitary sealable “tear-off stool collection pouch” that can be torn off the diaper and used for stool collection purposes in optional various models of the subject invention for medical purposes.

It is contemplated that as sensor technology improves and technology gets better a greater array of sensors will become available that will allow more diseases and conditions and variables to be sensed, detected and analyzed through the present invention. Furthermore, also, the size of the sensors will also become smaller allowing for more sensors to be incorporated into diaper models, or for sensor to become more comfortable, and probably at a lower cost. It is therefore a further object of the present invention to allow for the incorporation of these further sensor advancements into the technology presented.

A further object of the present invention is to provide structural support to allow removable and adjustably positionable anal, vaginal, and penile probe-type sensor(s) to be built into the diaper where a patient can wear the diaper with probes inserted into anal, vaginal, and or penile canals whereby the strapped-on diaper would assist in keeping the probes from being expelled from said anal, vaginal and penile canals from voluntary or involuntary muscular contraction(s).

Yet another object of the present invention is to provide a further embodiment of the present invention that will allow the construction of a sensor-type pad to be worn as a belt or to be tied to object(s) for sensing requirement(s).

Other objects and advantages of the present invention will become obvious to the reader and it is intended that these objects and advantages are within the scope of the present invention.

To the accomplishment of the above and related objects, this invention may be embodied in the form illustrated in the accompanying drawings, attention being called to the fact, however, that the drawings are illustrative only, and that changes may be made in the specific construction illustrated.

Other objects and advantages of the present invention will become apparent from the following descriptions, taken in connection with the accompanying drawings, wherein, by way of illustration and example, an embodiment of the present invention is disclosed.

In accordance with a preferred embodiment of the invention, there is disclosed a machine or device for hygiene and care requirements providing a more “Hygienic Diaper and or sensor pad And System With Alert Means For Treating And Caring For Wetness, Feces, & Disease” comprising: a disposable or re-useable diaper; a diaper with one or more “sensors” to detect and analyze ketone levels, sugar levels, blood, body fluids, liquids, gasses, drugs, bacteria, viruses, toxins or other agents; a “circuit card unit” connected to the diaper and connected to sensors in the diaper with a power source to drive circuits to alert caregivers when diaper becomes wet or when there is a bowel movement; a circuit and an antennae in the circuit card unit that will allow transmission means in the circuit system of the diaper to transfer sensor data findings and alerts of wetness and bowel movements to caregivers or medical staff; alert means to warn caregivers that wetness of the diaper or bowel movements have occurred on the diaper circuit system itself; algorithms to identify what sensors discover and report it to the caregivers on the “Nurse Remote Wand” and or directly to wireless receiver links computer database(s); algorithms and circuits to detect, analyze and report body temperature from sensors and or temperature probes from skin, from anal or vaginal or penile sensors or probes that are built into various diaper models; and identify a software interface to allow staff to input data relating to treatment plans, diagnoses, notes, and to track patient data which is editable and which has multiple security and access levels; a database where sensor information on each patient is dumped and stored from transmission(s) sent from diaper(s); a “Nurse Remote Wand” that receives sensor data through transmission means from the diaper's circuit system which also allows the data it receives to be downloaded to a computer patient treatment and planning database; an audio visual indicator for wetness and bowel movement alerts, and a LCD or LED readout to display what sensors have detected, and a “battery” or other power source to drive the “circuit card unit” on the diaper; an audio visual indicator for wetness and bowel movement alerts, and a LCD or LED readout to display what sensors have detected, and a “battery” or other power source to drive the “Nurse Remote Wand” unit; and “electrode snaps” and or other connection means to connect sensors to the “circuit card unit” of the diaper.

A very hygienic and highly advanced diaper is proposed with alert means for wetness, and for controlling contact with urine and feces & disease and for eliminating diaper rashes and other diseases and for controlling diseases that are detectable by analyzing anal, penile, and vaginal secretions, including analyzing urine and feces by analyzing the gasses and or the liquids expelled in the diaper. The invention is also designed to increase the comfort of infants, toddlers, children and older adults who may wear and use diapers and need them and is designed to improve sanitary conditions where weakened immune systems are characteristic in these age brackets where bacteria and viruses abound from anal, penile, and vaginal secretions and urine and feces.

The invention especially creates a more sanitary environment where caregivers will know immediately when urine and or feces have been expelled to correct the condition. The invention provides means to communicate to caregivers that a child or an adult under care may have urinated or expelled matter into their diapers so the care giver may change them to create a more sanitary condition as immediately as possible to comfort the patient or child to promote a healthier condition and to provide comfort and to relieve stress to the caregiver from crying or other disturbances that a child or patient may be verbalizing.

Another problem that this invention solves is that the invention can be used as a diagnostic tool at home or in hospitals or in nursing homes, day care centers or in other facilities. The proposed invention also allows the construction of different models, one for sensing and alerting the caregiver if the diaper has been filled with urine and or feces or secretions via a wetness sensor(s), and other models where sensors can be added to detect illness or other abnormalities where a sensor may detect blood in the urine, abnormal ketone levels, sugar levels, other abnormalities, impurities in the urine, and other dangerous conditions. A number of sensors can be used including liquid analysis sensors and gas-odor sensors that can be sandwiched in between the absorbent layers of the diaper material. Sensors can be placed in various positions throughout the diaper, either throughout the entire lining of the diaper, as a foil-type sensor, or other sensors, sandwiched between the absorbent layers 1 and 2, within multiple layers, or within an absorbent layer if there is one layer or directly under the permeable layer or in contact with the wearer of the diaper if the sensor can be woven into the diaper in a comfortable and in a hygienic fashion.

Different sensors can have different uses, to sense wetness, and also to analyze anal, vaginal or penile discharge and secretions for abnormalities, bacteria, viruses, and or disease. The inventive device includes a diaper preferably made out of disposable materials with absorbent layers with a wetness sensor and other sensors sandwiched between the layers of the diaper in the deluxe model. In the various models, there will be sensors in the lining of the diaper, composed of a foil lining, or other sensors, sandwiched between the absorbent layers of the diaper attached to a circuit to sense wetness, odor, disease, pathogens, bacteria, bacteria levels, viruses, or other abnormalities, that will alert the caregiver(s).

The “circuit card unit”, in the deluxe models for hospitals will have a transmission circuit that will transmit an ID for the diaper and that will alert the caregiver or nurse, on their remote wands, that a diaper is wet or that a disease or other abnormality has been sensed in deluxe models. Of course, various models can be marketed, one may be “wetness only detection” to detect whether urination has occurred, and or usually followed with feces expulsion, or other diaper models can be developed and marketed targeted to diagnose and sense for different conditions and diseases each to be manufactured with different sensors. The diapers can also be used in facilities as a method for stool collection. The Outer Shell Of The Disposable Diaper (Is Made Of A Plastic-Type Material). The Absorbent Layer(s) Of The Diaper. (The invention herein implies that it is understood that a diaper may be constructed with at least 1 absorbent layer to a maximum of “n” layers, and that sensors may be strategically embedded where appropriate to provide the benefits intended).

The Foil Wetness Lining Sensor(s) Or Other Sensors Are Designed To Sense Diseases, Parasites, Bacteria, Viruses, Blood, Blood Sugar Levels, Ketone Levels, Impurities, Toxins, Drugs, Body Fluids, Sebum, Secretions, Body Temperature, Or Other Detectable Items Of Interest. They Are Sandwiched Between Absorbent Layers Of Diaper Or They Are Positioned To Come Into Contact With The Skin. In the case of temperature probe sensors, such sensors are insertable into the anal, vaginal, and penile canals while the diaper being fastened upon the individual securely positions the temperature sensors in place to secure the temperature sensor(s) in their position. This also assists against muscular contractions that sometimes expel thermometers or probes out of anal or vaginal canals involuntarily or voluntarily.

(Furthermore, the invention herein implies that it is understood that a diaper may be constructed with just 1 absorbent layer to a maximum of “n” layers, and that sensors may be strategically embedded where appropriate to provide the benefit intended). Electrode Snaps Communicate From Sensors To Small Circuit Card Unit That Snaps On To Diaper and also operate to secure the circuit card unit to the diaper. Circuit Card Unit Senses Wetness, Disease, And Abnormalities, And Connects To Sensors. Other Sensors Designed To Detect Disease And Abnormalities Sandwiched Between Absorbent Layers Of The Diaper. (The invention herein implies that it is understood that a diaper may be constructed with just 1 absorbent layer to a maximum of “n” layers, and that sensors may be strategically embedded where appropriate to provide the benefit intended). The Circuit Card Unit Can Operate Via A Battery Or Via An AC Adapter For Hospitalized Patients Confined To Hospital Beds. The Permeable Lining Between The Infant Or Adult And “Absorbent Layer #1”, Designed To Allow Absorption Into The Absorbent Layers Of The Diapers. Nurse Remote Wand Is Used For Monitoring Deluxe Versions Of Diapers In Hospital Or Special Care Facilities Where There Are Multiple Patients And Sanitary Conditions Are To Be Observed. Sensors may be electronic or chemical where chemical sensors may change color or may provide other information when triggered.

The diaper as a further feature can have medication(s), salves, ointments, powder(s), aloe, healing agents, or other lotions chemically added to the diaper in various models in the absorbent layers or to other layer(s) of the diaper that come into contact with the wearer of the diaper for the treatment of diseases, abnormalities and or condition(s) associated with diaper rashes and or other diseases and condition(s) and abnormalities that can be treated by the wearing of a diaper through contact means or through transdermal transmission from the diaper to the skin.

Another feature of the diaper in deluxe models will allow the dumping of diaper patient data into computer files, databases, and for hard copy computer printouts on paper for the deluxe hospital models using the “nurse remote wands”. The “nurse remote wands” can be connected to a computer.

Another feature of the diaper is a built-in sanitary sealable “tear-off stool collection pouch” that can be torn off the diaper and used for stool collection purposes in optional various models of the subject invention for medical purposes,

Other features include handling patient data. The patient data can be transferred to a computer database and printouts can be printed on all the patients for progress reports that will give caregiver(s) and doctors information on the actual time of wetness, secretion, disease identification, abnormality identification, and or expulsion(s) and it will identify the condition(s) sensed by the sensor(s) and provide that information to the caregiver(s) and the doctor(s) in report form. The system will also come with software that will enable doctors(s), nurses and caregiver(s) to weigh a clean diaper and weigh a full diaper after expulsion(s) and or secretion(s) have been expelled or secreted into the diaper so that information can also be inputted into the record for each patient.

The Software provided will work automatically to combine information with a patient's Diaper ID number or identification that was programmed by caregivers and update records as time goes on with each diaper reset and cancellation when a new clean diaper is put on a patient. Graphing functionalities for the actual time of wetness, secretion, disease identification, abnormality identification, and or expulsion(s) into a diaper are built into the software as well as graphing functionalities for keeping track of the weight of a full diaper once matter has been expelled into the diaper, whether it is anal, penile, vaginal secretions or expulsions. Other features of the software will allow one to record the color of anal, penile, and vaginal secretion(s) and expulsion(s), their consistency, whether the patient had loose stools or diarrhea, and how the patient is responding to medication and to track progress. There is also a section where one can record notes for each patient in the record for each patient for each diaper change to track progress.

Another feature of the diaper through the “circuit card unit” will allow the clocking of the actual time when wetness or secretions are found by sensors, or when anal, penile or vaginal secretions are expelled into the diaper and have been sensed by the circuit card unit. It is obvious by this functionality that a clock circuit will be included in the circuit card unit that keeps track of the time for this feature that will store the actual time of such expulsion(s) into the memory of the circuit card unit of the diaper and that a caregiver can scroll on the “circuit card unit” through the messages on the “LCD” display to check for the time of expulsion into the diaper.

In the deluxe version of the “circuit card units” for the hospital units, the caregiver can see this information on the “nurse remote wand” as well because the “actual time” will be transmitted immediately once wetness, secretion, disease identification, abnormality identification, and or expulsion occurs, and the “circuit card unit” will also display the same information that is attached to the diaper of the patient. On home units no “nurse remote wands” are used, but caregiver(s) can scroll through messages on the LCD to view the actual time of wetness, secretion, disease identification, abnormality identification, and or expulsion(s) as home units still have the same functionalities except for the “nurse remote wand” capability.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. 1 is a perspective view of the invention illustrating a disposable or reusable diaper with sensors and the circuit card unit in place.

FIG. 2 is a fragmentary perspective view of the diaper illustrating the diaper with the “circuit card unit” removed and the sensor(s) “3” and “5”.

FIG. 3 illustrates the diaper in a vertical cross-section 3-3 taken from FIG. 2 with a detachable water-tight plastic and sealable “tear-off sanitary stool-urine-materials collection pouch” attached to the diaper through detachable-attachment means.

FIG. 4 is a perspective view illustrating the detachable watertight plastic and sealable “tear-off sanitary stool-urine-materials collection pouch” as detached from the diaper through detachable-attachment means.

FIG. 5 is a vertical cross sectional view of the diaper system similar to FIG. 3 with removable and adjustable skin temperature or disease sensors and or temperature or disease sensors or probes designed to be inserted into the anal, vaginal, and or penile canals for temperature or disease readings having sensors or probes designed to be in their functional position to take readings while a person wears the diaper.

FIGS. 5(A) and 5(B) depict an enlarged fragmentary perspective view of FIG. 5 illustrating the removable and adjustable anal sensor, the vaginal sensor, and the penile sensor, each comprising a removable sensor stem probe section; a movable socket “head” held to move in a bi-directional manner.

FIG. 5(A)(A) is a top plan cross sectional view taken from FIG. 5(A) showing the sensor sockets movably sliding in a bi-directional manner and secured to remain within a pocket groove.

FIG. 5(B) is a perspective view illustrating an alternate model of the diaper where the penile sensor may have its distance extended from the diaper by utilizing a penile probe having a wire of “x” length with its end terminated with a wired connector connecting to the diaper at a socket or a wire contact location and where the penile sensor can be exchanged with other sensor(s) for other sensing needs that can be used for other detection requirements.

FIG. 5(C) is a perspective view illustrating a detachable sensor probe stem fittable to a socket head of the diaper attachable to the socket through screw-thread means.

FIG. 5(D) is a perspective view illustrating a detachable sensor probe stem fittable to a socket head of the diaper attachable to the socket through snap-fit means.

FIG. 6 is a plan view of the invention of the deluxe version of the “circuit card unit” for hospital, nursing home or day care use having patient identification functionality and transmission means to send sensor data to from the “circuit card unit” to computers and to the “nurse remote wand”.

FIG. 7 is a plan view of the invention of the “circuit card unit” for home use.

FIG. 8 is a rear-elevational view of the invention of the “circuit card unit” both for home use and for hospital, nursing home or day care use showing how the “circuit card unit” connects to sensors through “electrode snaps” or through “connection” means.

FIG. 9 is a plan view of the invention of the “Nurse Remote Wand” unit for hospital, nursing home or day care use.

FIG. 10 is a plan view of the invention of the “Nurse Remote Wand” unit for hospital, nursing home or day care use with an optional magnetic card reader for reading patient identification cards with magnetic stripes.

FIG. 11 is a plan view of the invention of the “Nurse Remote Wand” unit for hospital, nursing home or day care use with a jack for attaching an optional magnetic card reader attachment for reading patient identification cards with magnetic stripes.

FIG. 12 is a schematic block diagram view of the deluxe hospital “circuit card unit” of the invention showing internal operating circuits including sensing, reporting, transmission, patient identification functionality, and other features.

FIG. 13 is a schematic block diagram view of the non-deluxe home or consumer version of the “circuit card unit” of the invention showing a “circuit card unit” without transmission functionality and without patient identification functionality.

FIG. 14 is a schematic block diagram view of the “nurse remote wand” of the invention without a magnetic card reader.

FIG. 15 is a schematic block diagram view of the “nurse remote wand” of the invention with a magnetic card reader.

FIG. 16 is a schematic block diagram view of the “nurse remote wand” of the invention with a jack for an attachable magnetic card reader as an accessory item.

FIG. 17 is a flow chart diagram view depicting a software-database system and proposed tables.

FIG. 18 is a plan view of an alternate embodiment of the present invention showing a sensing pad (as opposed to a diaper) of “X” length and “Y” width” having an absorbent layer embedded with one or more sensor(s) that can be applied to a wound or to contaminated areas, or to spills, for sensing detection and reporting application applications the same way the diaper works, to be connected through electrode snaps to the “circuit card unit(s)”, as provided in the present invention.

FIG. 18(A) is a plan view of an alternate embodiment of the present invention showing a sensing pad (as opposed to a diaper) of “X” length and “Y” width” having an absorbent layer embedded with one or more sensor(s) that can be applied to areas as a belt for sensing detection and reporting application applications the same way the diaper works, to be connected through electrode snaps to the “circuit card unit(s)”, as provided in the present invention.

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

### DETAILED DESCRIPTION OF THE INVENTION

Turning now descriptively to the drawings, in which similar reference characters denote similar elements throughout the several views, the attached figures illustrate a hygienic diaper and or sensor pad with alert means for wetness, feces & disease, which comprises a diaper preferably made out of disposable materials with absorbent layers with a wetness sensor and other sensors sandwiched between the layers of the diaper in the deluxe model. A diaper is herein described that will provide greater hygiene for the wearer while providing means for treating, for providing medical diagnoses through sensor means and providing greater care through electronics and software-database patient downloads of sensor derived data gathered from diaper(s).

In the various models, there will be sensors in the lining of the diaper, composed of a foil lining, or other sensors, sandwiched between the absorbent layers of the diaper attached to a circuit to sense wetness, odor, disease, bacteria, viruses, blood, ketone levels, sugar levels, or other abnormalities, that will alert the caregiver(s). The circuit, in the deluxe models for hospitals will have a transmission circuit that will transmit an ID for the diaper and that will alert the caregiver or nurse, on their remote wands, that a diaper is wet or that a disease or other abnormality has been sensed in deluxe models. It has been further described that the sensor(s) of the diaper can be used to sense and detect or analyze ketone levels, sugar levels, blood, body fluids, bacteria, viruses, toxins, drugs, skin conditions, other abnormalities, or other things that can be sensed through sensor technologies that can be used in disposable or re-usable diapers utilized in this invention.

Of course, various models can be marketed, one may be wetness only to detect whether urination has occurred, and or usually followed with feces expulsion, or other diaper models can be developed and marketed targeted to diagnose and sense for different conditions and diseases each to be manufactured with different sensors (one-time use, insertable, re-useable, sterilizable, and or throw-away or disposable). The diapers can also be used in facilities as a method for stool collection.

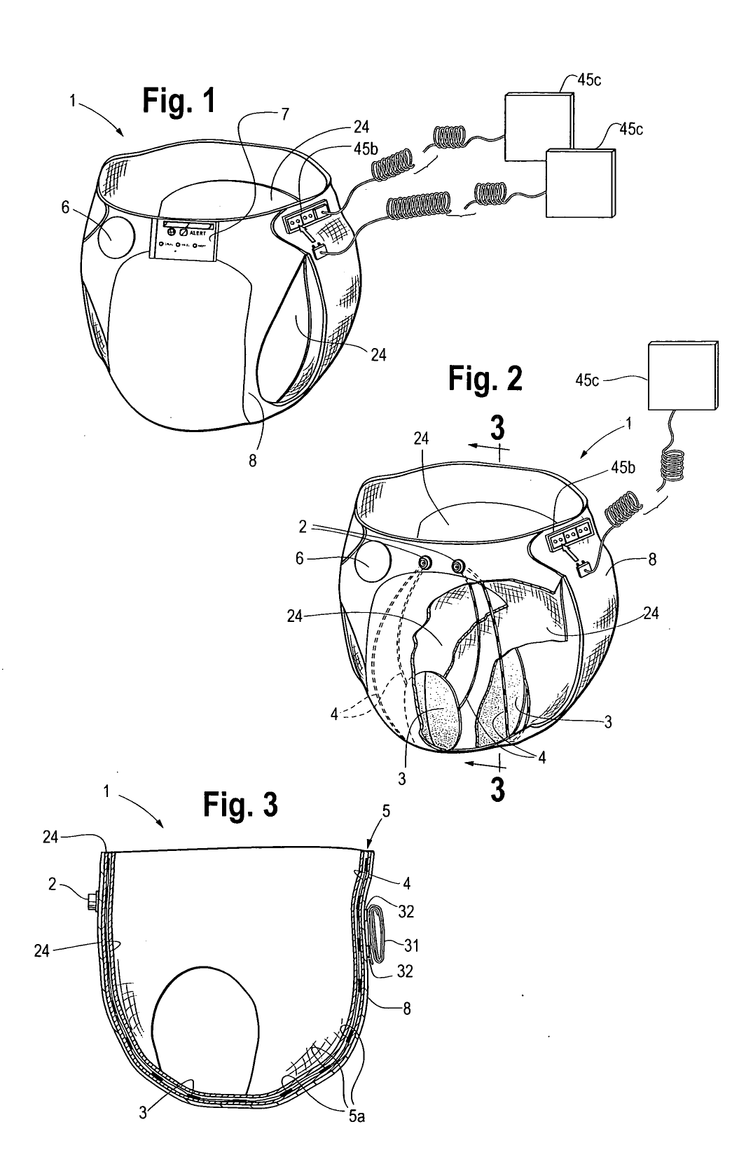

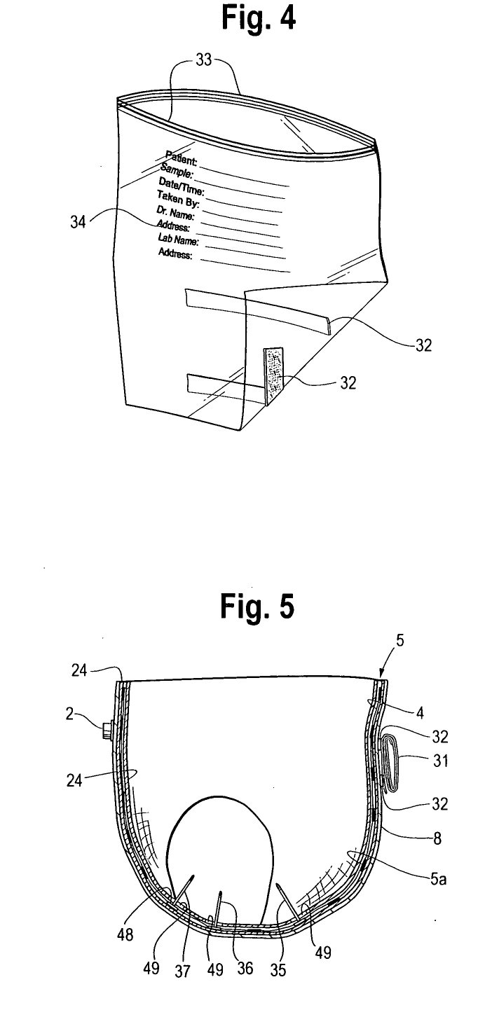

FIGS. 1, 2, and 3 depict a more advanced reusable or disposable hygienic diaper and or sensor pad system with wetness and bowel movement alert means and for treating disease. These embodiments incorporate the following basic components: an advanced hygienic diaper 1 for infant, toddler, child, or adult use; “n” number of “electrode snaps” 2 or connection means 2 stitched into the absorbent layers of the diaper connecting to “circuit card unit” 7 and sensor(s) 5; sensors connected through foil or wire connections 4 to the “circuit card unit” through absorbent layer(s) of “n” number 24, also showing one or more sensors 5(a) placed in the diaper 1, with “n” number of electronic sensor areas 5 on the diaper where sensors can be placed strategically on the diaper and embedded in absorbent areas of the diaper or in direct contact with the skin of the wearer of the diaper 5; as more clearly shown in the cut-away view of the absorbent layer(s) of the diaper showing the sensor(s) “3” connecting to the “circuit card unit” 7, through wires or electrodes 4 leading to electrode snaps 2 which is attached to the diaper through attachment means preferably somewhere along the waistline of the diaper; one or more optional sensor connector(s) 45(b) each designed to insert one or more sensor leads to accommodate one or more optional sensor(s) 45(c) in them; and a non-permeable or semipermeable outer shell or lining of the diaper 8. Please note that different sensors can be used including but not limited to one or more electrochemical type sensor indicators that can display color change 6 and or that can provide an electrical signals to the “circuit card unit” that can also be incorporated into the diaper design. The diaper can be constructed in different models with one or more sensors combining both chemical and electronic sensors or diapers may be developed just having solely chemical or solely electronic sensors and they can be placed in strategic areas throughout the diaper as desired. The visual/audible indicators, whether they are electronic or chemical indicators, can also be placed in different areas than shown. FIGS. 3 and 4 also illustrate an optional feature on some models of our present invention of a detachable water-tight plastic and sealable “tear-off sanitary stool-urine-materials collection pouch” 31 attached to the diaper through detachable-attachment means 32, having a sanitary water-tight sealable closure means 33 to contain urine, stool, blood, secretion and or material samples, and further having a writable surface area on it 34 to record notes such as patient information or lab notes upon the pouch.

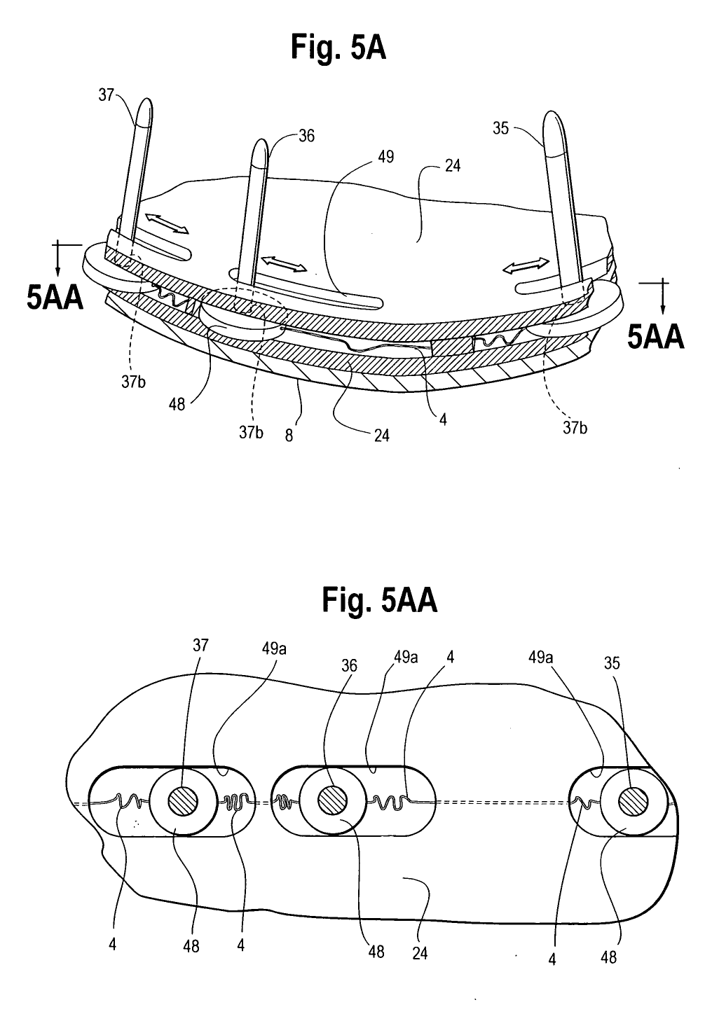

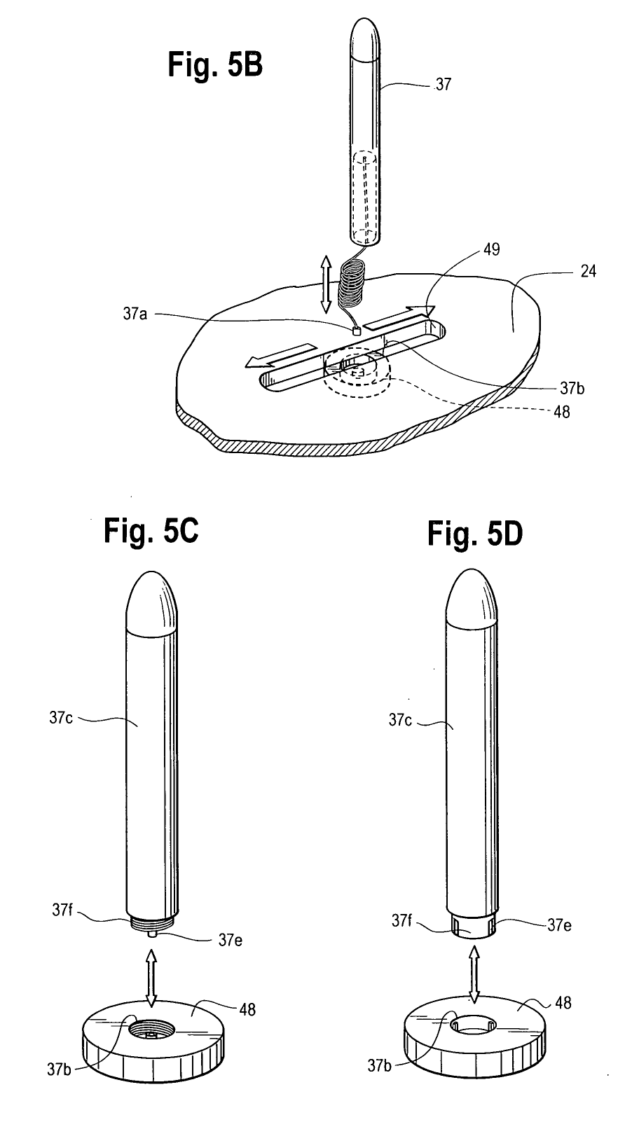

FIG. 5 depicts a cross section of the diaper with removable and adjustable skin temperature or disease sensor(s) and or temperature or disease sensor(s) mounted in the diaper 1 in fixed and or adjustable strategic position(s) with flexibility having at least three comfortable flexible sensor(s): an anal sensor 35 of “x” length and “y” thickness, a vaginal sensor 36 of “x” length and “y” thickness, and or a penile sensor 37 of “x” length and “y” thickness [where “x” and “y” can be the same and or different number(s) for any and all sensor(s)], to be positioned and inserted into anal, vaginal, and penile canals respectively, where upon the wearer being fit with the diaper with sensors in place wearing the diaper prevents the sensors from being expelled from voluntary or involuntary muscular contraction(s) by virtue of holding the sensors securely into place after insertion by the patient wearing the diaper; whereby all sensor(s) are connected through “n” number of electrodes or wires to “n” number of electrode snaps whereby the “circuit card unit” can be attached; and where the diaper has movable socket “heads” 48 for the anal, vaginal and or the penile sensor(s); where said sockets are sandwiched between upper absorbent layer 24 and lower absorbent layer 24 and held to move in a bi-directional manner in a groove pocket 49 that has enough absorbent material cutout from the diaper to allow the socket to move back and forth in a bi-directional manner (but not to allow the sensor(s) to escape into the rest of the diaper) between the absorbent layer(s) 24 said socket “heads” having a penile sensor stem probe 37, a vaginal sensor stem probe 36 and or an anal probe stem 35 that can be removed or attached into the socket “heads” through snap-fit means or screw-fit means for connecting sensor stem probes; where socket “head(s)” are designed to slide bi-directionally within groove(s) 49.

FIGS. 5(A) and 5(A)(A) depict an enlarged fragmentary perspective view of FIG. 5 illustrating the removable and adjustable anal sensor 35, the vaginal sensor 36, and the penile sensor 37, each comprising a removable sensor stem probe section 37(c) (as shown in FIG. 5C and FIG. 5D); a movable socket “head” 48; where said socket is sandwiched between upper absorbent layer 24 and lower absorbent layer 24 and held to move in a bi-directional manner in a groove pocket 49(a) that has enough absorbent material cutout from the diaper to allow the socket to move back and forth in a bi-directional manner (but not to allow the sensor(s) to escape into the rest of the diaper) between the absorbent layer(s) 24 said socket “heads” having a wire contact location 37(b) within socket “heads” for connecting sensor stem probes; a socket “head(s)” which are designed to slide bi-directionally within groove(s) 49 and where each sensor stem probe has electrical connectors 37(f) (shown in FIGS. 5C and 5D) to mate with electrical connections within said socket “head(s)” in the wire contact location 37(b) (shown in FIGS. 5A, 5C and 5D); stems having screw and or snap-fit connection means 37(e) (also shown in FIGS. 5C and 5D) so that sensor stem(s) and their electronic connections 37(f) on the ends of the sensor stem(s) 37(c) can be snapped-in and or turned into place securely or removed into the anchor “head” or socket/wire contact location 37(b) for sensing applications; wires or electrodes proceeding from the socket to adjoining sensor(s) proceeding to “electrode snaps” 2 that connect to the circuit card unit 7 (as is illustrated in FIGS. 1, 6, 7, and 8), all with anal, vaginal, and penile sensor anchor heads 48 sandwiched and secured underneath and between the upper absorbent layer 24 and the lower absorbent layer 24 of the diaper with the sensing stem probe portion of the sensor(s) protruding inwardly through the absorbent layers of the diaper through groove(s) 49 so that they may be easily moved and adjusted to be inserted into the anal, vaginal or penile canal(s), and whereby the sensors may be slid bi-directionally in groove(s) 49 by moving the sensing portion(s) of the sensors by grasping and moving them within said groove(s) 49 within the absorbent layer(s) of the diaper to position and insert the sensor(s) into the anal, vaginal, and penile canals respectively in a comfortably fitting manner, and whereby the anchor “heads” 48 of the sensor(s) are large enough and tight-fitting enough with some resistance within said groove(s) 49 that once adjusted they remain in position, and where the sensor(s) remain supported in their upright position(s) within the layers of the fabric of the diaper where the sensors remains supported in their functional flexible position(s) for a comfortable fit or insertion into their anal, vaginal or penile canals for temperature, disease, or other detection interests.

FIG. 5(B) depicts an alternate model of the diaper where the penile sensor may have its distance extended from the diaper by utilizing a penile probe 37 having a wire of “x” length with its end terminated with a wired connector 37a connecting to the diaper at a socket or a wire contact location 37b within the socket “head” 48 accessible through a groove 49 and where the penile sensor can be exchanged with other types of sensor(s) for other sensing needs that can be used for other detection requirements.

FIG. 5(A) illustrates sensor head(s) 48 that are designed to slide bi-directionally within one continuous groove in optional embodiments of the diaper in alternate models of the diaper or in separate groove(s) 49 as is illustrated in this drawing. It is assumed that separate grooves as indicated in the drawing would give more support for individual sensor(s) and would still allow for adjustment. It is further contemplated and shown that in the case of the penile sensor 37 (as illustrated in FIG. 5B), the penile sensor probe may have its distance extended from the diaper by utilizing a penile probe having a wire of “x” length with its end terminated with a wired connector 37(a) connecting to the diaper at a socket or a wire contact location 37(b) positioned or located to be movable bi-directionally within the penile groove 49 where the penile sensor can be attached to the diaper and where the penile sensor may then be placed manually into the penile canal to accommodate the easy placement of the penile sensor into the penile canal. Please note that the wired version of the penile sensor connector can also be removed or detached from the socket as desired and replaced with other types of sensor(s) with extendable wires such that can be run and placed unto different parts of the body to measure different things such as respiration, heartbeat, pulse, skin condition, bleeding, sinus trouble or wheezing, or other detection needs as mentioned, or as may be desired. The other sensor(s) including the anal and the vaginal sensor(s) can also be designed to operate in the same way and in alternate models of the diaper it may be fashioned to have a plurality of sensor(s) or one or more of such sensors in addition to the anal, vaginal, or penile sensor(s) or in lieu of anal, vaginal or penile sensor(s) or designed to be incorporated in absence of said anal, vaginal or penile sensor(s) where there are “x” number of sensor(s) with extendable wires connected to the diaper that can be used for detection requirements.

FIG. 5D depicts a sensor probe stem 37c with electronic connection means 37f and snap-fit means 37e, said electronic connection means connecting to an electronic connection 37b in the socket head 48.

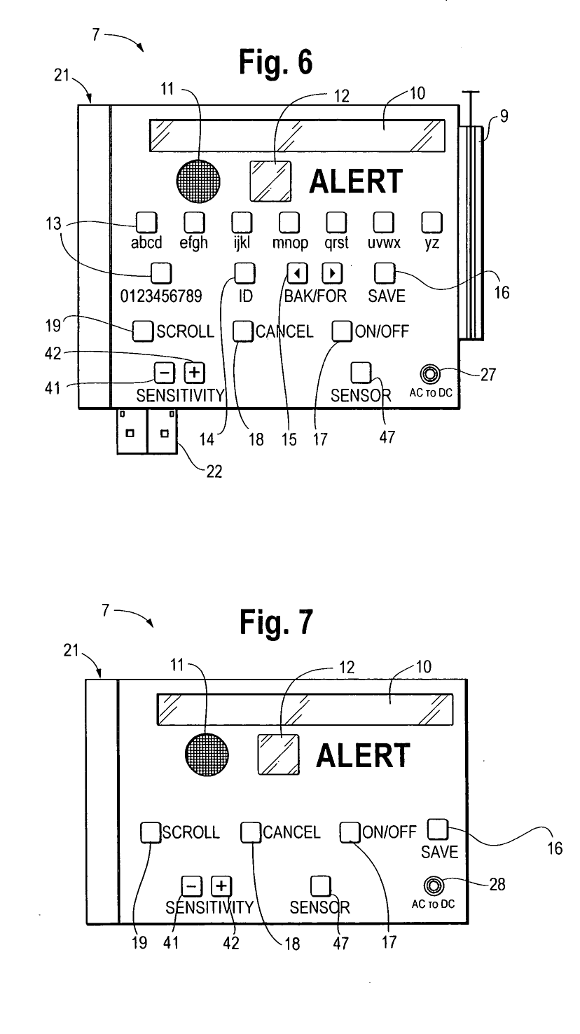

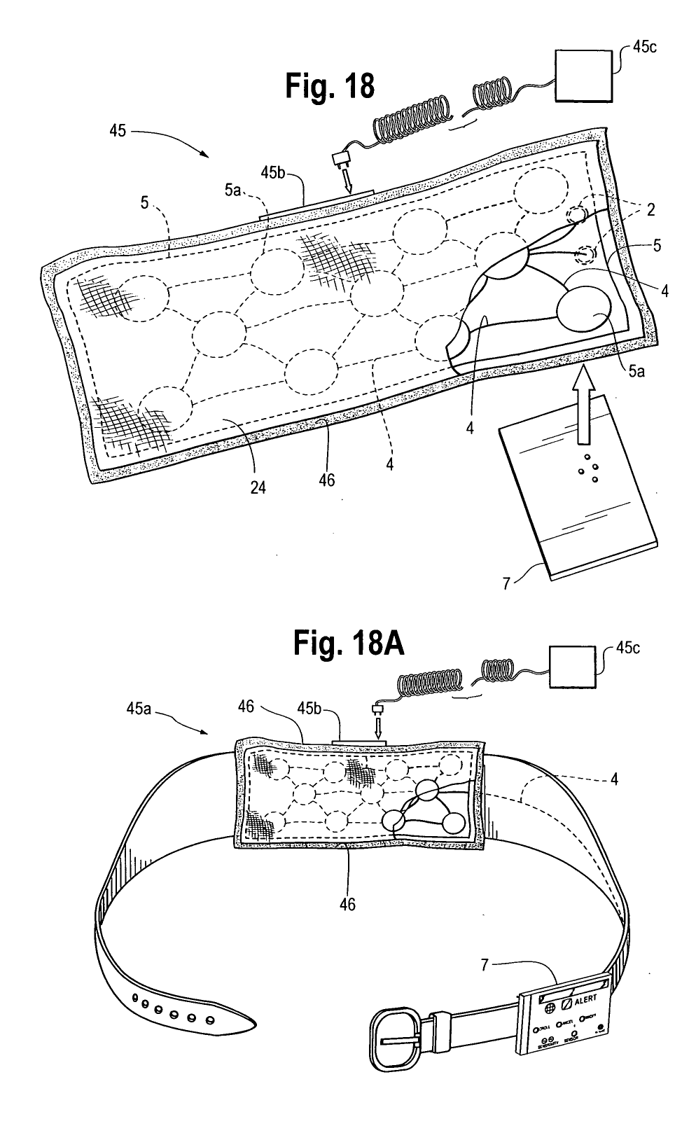

FIG. 6 depicts a more advanced reusable or disposable hygienic diaper system with wetness and bowel movement alert means and for treating disease. This embodiment incorporates the following basic components: a deluxe hospital “circuit card unit” 7 containing circuitry for analyzing and reporting sensor data findings on the readout panel 10 of the “circuit card unit” of the diaper system discovered in the diaper and further transmitting said sensor data to a “nurse remote wand” or directly to computer(s); an antennae 9 for transmitting said sensor data along with patient identification data; an LCD, LED or other readout means 10 for wetness or bowel movement alerts and sensor reading findings built into the “circuit card unit” 7; a speaker for audible alerts 11; a bright flashing LED alert indicator 12 for alerting caregivers of diaper wetness or bowel movements; an ID push button program mode button 14 to enter program identification mode, or to display the current patient ID, or none “as default”; push buttons or other interface means “A-Z”, “0-9” as shown 13 that can be selected by depressing said push buttons repeatedly to enter patient identification data; when push buttons are pushed the “circuit card unit” 7 prompts the caregiver to enter patient identification information which then allows the caregiver to enter an ID for the specific patient diaper which can then be transmitted along with the type of alert to the “nurse remote wand” of the caregiver or to computers; backward/forward push button(s) 15 allow the caregiver to enter or edit letters or numbers displayed in the readout when entering a patient identification in program mode for editing purposes to back-up or go forward on the readout panel and correct or edit mistakes on the identification of the patient before saving the patient identification for the specific diaper “circuit card unit”; a “save button” 16 for saving the patient identification entered through the push buttons into the circuit card unit after it is entered successfully by selecting desired push buttons; two buttons (−) 41 and (+) 42 to adjust the “sensitivity” of the sensors to make them less or more sensitive respectively; pre-programmed means are achieved through hard-wired circuitry in the circuit card unit having the functionality to be able to turn sensor(s) on or off selectively on the circuit card unit(s) to pinpoint what area(s) should be sensed by depressing “sensor select” button 47 to select a desired sensor, once a desired sensor is selected that sensor location will be displayed on the circuit card unit's readout, depressing the “cancel button” within 5 seconds of the location of the sensor location being displayed on the readout will toggle that sensor on or off; the circuit card unit will automatically identify the sensor(s) with an area ID so when they report their data they also report their sensor location, as their sensor ID, and or the area they are sensing to help assist data gathering information and analyses needs whereby this feature shall apply to all embodiments of the present invention and models of the deluxe “circuit card unit” for the diaper version of the present invention and to the “sensing pad” and the “sensing pad belt” versions of the present invention (shown in FIG. 18) by depressing button 47 to select sensor(s). For each diaper or sensor pad there is a Master ID for each Diaper and or Sensor Pad versions of the present invention whereby the type and location of each sensor are programmed into the attached circuit card unit allowing not only sensor reading findings data but also location data of the sensor and the type of sensor being used as information to be provided with each transmission of the data event. Each sensor and each location on the diaper or sensor pad will have its own ID and or code and these will be coded into the master ID of the diaper or sensor pad; furthermore, the “sensor select” button 47 can be depressed continually to select sensors to selectively turn them on or off as described, however when cycling through the list of sensor(s) another feature of the “sensor select” button will be to enter the circuit card unit into a mode to program the internal clock of the “circuit card unit” whereby the user can enter the current time, and date and specify “A.M.” or “P.M.” utilizing the patient identification push buttons 13 to enter said time and date information; an on/off or power button 17 for powering the “circuit card unit” and also used to reset the unit and to clear the “circuit card unit's” memory from sensor findings; a cancel button 18 for cancelling an operation or an alert; a scroll button 19 to allow caregivers and or medical staff to scroll through alerts and sensor findings on the circuit card unit to see whether or not it is for wetness, bowel movements, disease, drugs, or some other impurity that sensors have sensed; a USB port or other computer connection 22 to download sensor finding data to computer(s); a battery compartment and or battery 21, and or a power source via an AC adapter 27 to provide power to the “circuit card unit” 7.

FIG. 7 depicts a more advanced reusable or disposable hygienic diaper system with wetness and bowel movement alert means and for treating disease. This embodiment incorporates the following basic components: a non-deluxe home or consumer version of the “circuit card unit” 7 containing circuitry for analyzing and reporting sensor data findings discovered in the diaper and reporting them on the circuit card unit's readout panel 10; an LCD, LED or other readout means 10 for wetness or bowel movement alerts and sensor reading findings built into the “circuit card unit” 7; a speaker for audible alerts 11; a bright flashing LED alert indicator 12 for alerting caregivers of diaper wetness or bowel movements; an on/off or power button 17 for powering the “circuit card unit” and also used to reset the unit and to clear the “circuit card unit's” memory from sensor findings; a cancel button 18 for cancelling an operation or an alert; a scroll button 19 to allow caregivers and or medical staff to scroll through alerts and sensor findings on the circuit card unit to see whether or not it is for wetness, bowel movements, disease, drugs, or some other impurity that sensors have sensed; two buttons (−)41 and (+)42 to adjust the sensitivity of the sensors to make them less or more sensitive respectively; a battery compartment and or battery 21, and or a power source via an AC adapter 28 to provide power to the “circuit card unit” 7.

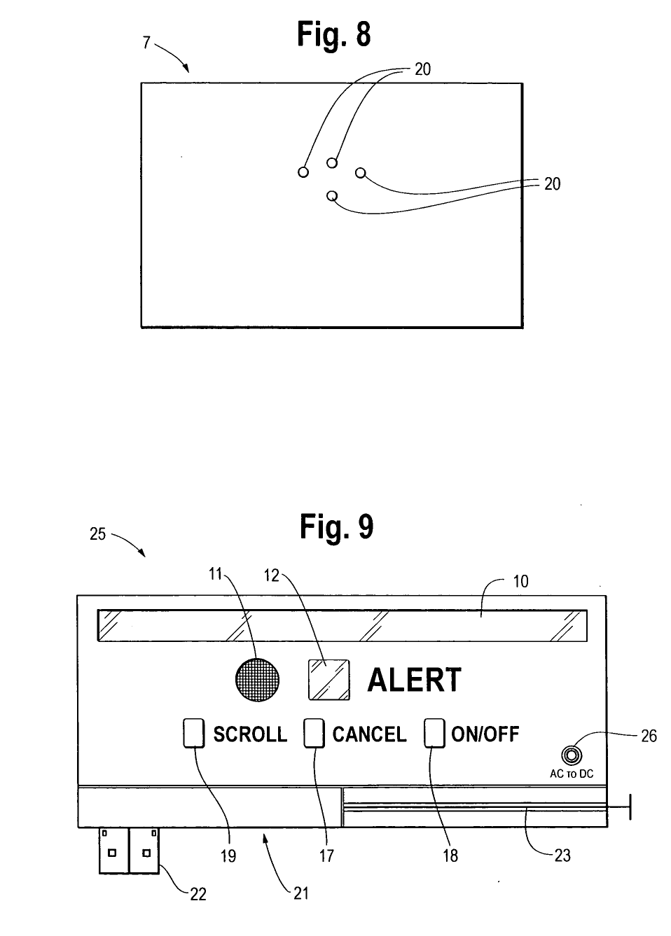

FIG. 8 depicts a more advanced reusable or disposable hygienic diaper system with wetness and bowel movement alert means and for treating disease. This embodiment incorporates the following basic components: a back view of the “circuit card unit” 7 is shown exposing “n” number of “electrode snaps” or sensor connections 20 whereby the “circuit card unit” 7 may be connected and secured to the diaper as is illustrated, turning back to FIGS. 1, 2, and 3 where the “circuit card unit” secures itself to electrode snaps labelled “2”, as depicted, where item 7 in turn is connected to “n” number of electrodes leading to “n” number of sensor(s) throughout the diaper; powered by batteries or a power source 21. Please note that FIG. 8 applies to home versions and to deluxe hospital versions of the “circuit card units” as depicted in both FIG. 6 and FIG. 7.

FIG. 9 depicts a more advanced reusable or disposable hygienic diaper system with wetness and bowel movement alert means and for treating disease. This embodiment incorporates the following basic components: a “nurse remote wand” for deluxe hospital models 25 that receives sensor findings and wetness and bowel movement alerts from the “circuit card unit(s)” 7 (shown in FIGS. 1, 6, 7 and 8); a LCD, LED or other readout means 10 on the “nurse remote wand” 25 for wetness, disease and or abnormality alerts where the readout will display if the diaper is wet, and if a disease, bacteria, virus or other abnormality is picked up by a sensor that is utilized, and it will display the diaper identification number; a speaker 11 for audible alerts; a bright flashing LED alert indicator 12 for alerting caregivers of diaper wetness or bowel movements; an antennae 23 to receive sensor findings, wetness and or bowel movement alerts from circuit card unit(s) from single or multiple patient(s); an on/off or power button 17 for powering the “circuit card unit” and also used to reset the unit and to clear the “circuit card unit's” memory from sensor findings; a cancel button 18 for cancelling an operation or an alert; a scroll button 19 to allow caregivers and or medical staff to scroll through alerts and sensor findings on the circuit card unit to see whether or not it is for wetness, bowel movements, disease, drugs, or some other impurity that sensors have sensed; a computer connection 22 to upload sensor findings data, patient identification, times of urination of fecal expulsion or other sensor detections and or wetness and bowel movement alerts received from “circuit card unit(s)” to a computer or to programmable digital assistants and or to databases; a battery compartment and or battery 21, and or a power source via an AC adapter 26 to provide power to the “circuit card unit” 7 (as illustrated in FIGS. 1, 6, 7, and 8).

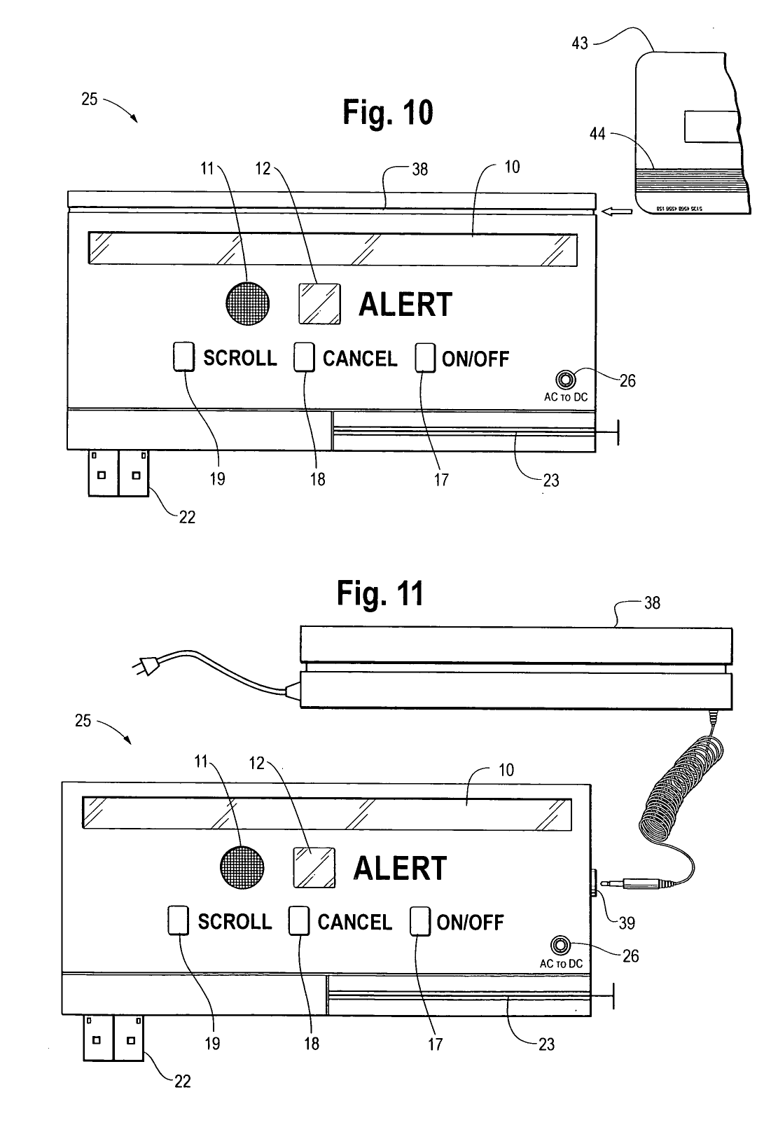

FIG. 10 depicts an optional version model of the “nurse remote wand” having all the components of FIG. 9 having an additional optional magnetic card reader 38, built into the “nurse remote wand” to scan and read magnetic patient identification cards 43, having a magnetic stripe 44, and to store patient information along with sensor data collected from diaper(s) into the “nurse remote wand” which can later be downloaded to computer(s).

FIG. 11 depicts yet another version of the “nurse remote wand” having again all the components of FIG. 9 with an optional magnetic card reader jack 39, built into the “nurse remote wand” to enable the purchase of a magnetic card reader 38 as an accessory item to be added to the “nurse remote wand” for scanning and reading magnetic patient identification cards and to store patient information along with sensor data collected from diaper(s) into the “nurse remote wand” which can later be downloaded to computer(s).

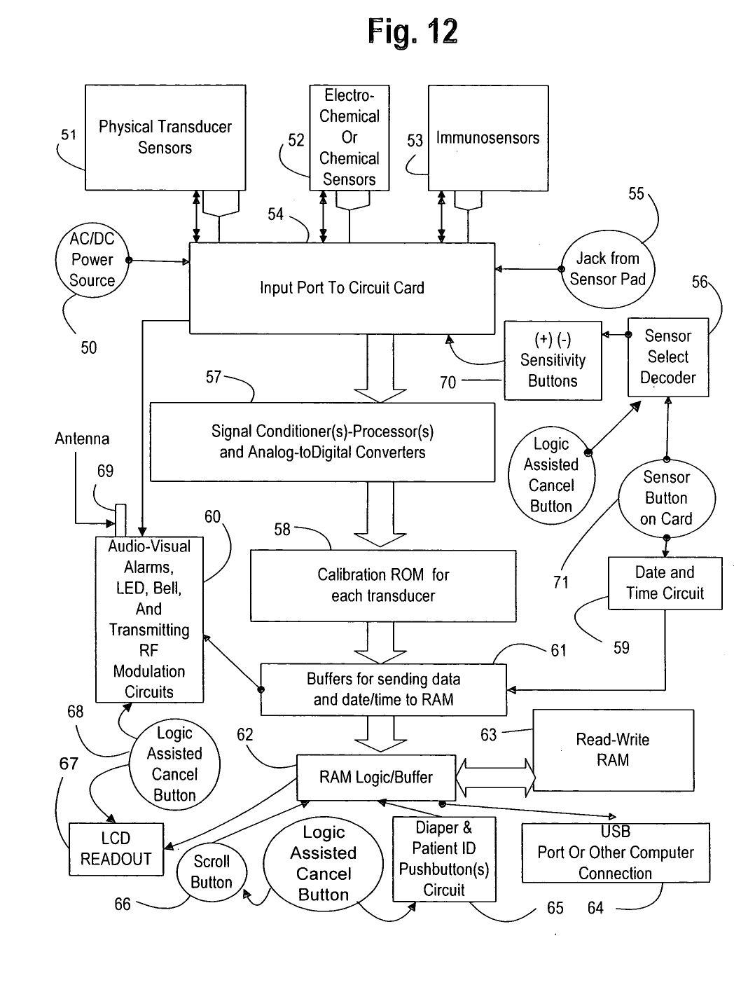

FIG. 12 depicts a more advanced reusable or disposable hygienic diaper, sensor pad and or sensor belt system with wetness and bowel movement alert means and for sensing and treating disease. This embodiment incorporates the following basic components: a schematic block diagram view of the “deluxe circuit card unit” for hospital, day care and nursing home use where it is desired to have automated readings of sensor finding(s).

Controllable Functions:

The circuit card is powered by a DC battery and has the option to be plugged into an AC outlet for longer use on non-ambulatory patients. The Scroll (sensor) button 71, on the face of the card, selects the transducers mounted on the diaper and has the ability to allow the user to turn them on or off selectively by stepping through them. The sensitivity selector on the card 70, adjusts the gain and sensitivity of the sensors for individual patients. Patient identification data, and diaper identification is input to the memory with the push buttons 65 and sensor findings can be scrolled through the scroll button on the card 66. The date and time are also selectable by the programmable push buttons 65 to set the date and time through the date/time circuit 59, which is used to annotate the data when a discharge or “finding of interest” is detected. The audio, visual and transmission alarms including an RF or radio frequency modulation signals circuit 60 are also selected by the Scroll (sensor) button 71 working through a sensor select decoder 56 and cleared by a LOGIC ASSISTED CANCEL button 68 that is used to perform different functions through logic throughout the “circuit card unit”. The card is also shown to have an optional USB or other computer connector or port that will allow the circuit card unit to dump its sensor finding(s) information to computer(s) directly without the use of an antenna.

Furthermore, FIG. 12 is comprised of the following items:

Description of Specific Circuit Blocks:

50. DC power source with AC adapter—the battery is connected to the card and the AC selector is attached to a bridge-filter circuit to convert the AC voltage to a DC level suitable for operation of the circuit card.

51-53. The sensor circuits are divided into three regions to accommodate the different electronic requirements to process and decode the signals arising from each.

51. Physical transducer/sensors include variables such as temperature and humidity (wetness). They are attached to the sensitivity selector through the input port (54) that adjusts the gain on each through a potentiometer. Outputs from the sensors go to signal conditioning circuits (57), including analog-to-digital conversion. The processed signal goes to a calibration circuit (58) for conversion into digitized interpreted data for storage and transmission. A digital signal present at the signal conversion circuit output activates a series of AND gates to pass the digitized sensor data with digital date and time information to the memory. Simultaneously, an activation signal is sent to the enabled alarms.

52. Electro-chemical and or chemical sensors include variables such as sugar levels, ketone levels, blood and other chemicals related to medical conditions, or chemical warfare. They are attached to the sensitivity selector through the input port (54) that adjusts the gain on each by a potentiometer. Outputs from the sensors go to signal conditioning circuits (57), including analog-to-digital conversion. The processed signal goes to a calibration circuit (58) for conversion into interpreted data for storage and transmission.

A digital signal present at the signal conversion circuit output activates a series of AND gates to pass the digitized sensor data with digital date and time information to the memory. Simultaneously, an activation signal is sent to the enabled alarms.

53. Immunosensors include variables such as bacterial and viral concentrations, toxins, parasites and emissions related to skin conditions, or to bacteriological or viral warfare. They are attached to the sensitivity selector through the input port (54) that adjusts the gain on each by a potentiometer. Outputs from the sensors go to signal conditioning circuits (57), including analog-to-digital conversion. The processed signal goes to a calibration circuit (58) for conversion into interpreted data for storage and transmission. A digital signal present at the signal conversion circuit output activates a series of AND gates to pass the digitized sensor data with digital date and time information to the memory. Simultaneously, an activation signal is sent to the enabled alarms.

54. Input Port To Circuit Card.

55. Jack From Sensor Pad. The jack from the sensor pad takes the input from the sensors on the pad to the input port using identical buffers and logic as from the sensors on the diaper.

56. Sensor Select Decoder. The decoder circuit allows scrolling through each mounted sensor. When one is selected, a flip-flop will be set to enable or disable that sensor in response to the CLEAR button that turns the selected sensor off.

A timing circuit in the port allows clearing the selected sensor to be disabled within 5 seconds of selecting the sensor. Otherwise the sensor will remain active.

57. Signal Conditioner(s)-Processor(s) and Analog-to-Digital Converters. The signal conditioners and processors amplify, filter and convert the signals from the sensors to digital format. These are sent to the calibration Read-only-memory (ROM) for processing.

58. Calibration ROM For Each Transducer. The calibration Read-only-memory (ROM) contains the calibration code algorithm to convert the conditioned signal data to usable data, such as specific temperature or humidity units. This digital data can then be stored in RAM, used to set off a selected set of alarms or appear on the LCD readout on the card.

59. Date & Time Circuit. The date and time circuit can be set with the Scroll (sensor) button on the card. It contains an internal clock and buffers to store date and time digitally. These are temporarily stored in the data buffers (61) to be sent to memory or the LCD readout when sensor data are present to tag sensor data finding(s) and stamp them with a time and date along with a patient/diaper/sensor type identification/and sensor area location identification (to specify what each sensor plugged into the circuit card unit is sensing).

60. Audio-Visual Alarms, LED, Bell, & Transmitting RF Modulation Circuits. The alarm system consists of audio, visual and transmission alarms. They are activated by the Scroll (sensor) button and cleared by the CANCEL button. The audio alarm is a bell, the visual alarm is a light emitting diode (LED) and the transmission alarm is transmitted to the Nurse Remote Wand or directly to the nurses' station computer(s) by the attached antenna (69). A decoder sends the alarm information to the LCD display for readout.

61. Buffers For Sending Data And Date/Time To RAM. The data buffers collect the calibrated digital data from the Calibration ROM (58) and the Date/Time circuit (59) and send the information to the selected alarms (60), the LCD readouts (67) and the RAM buffer for storage.

62. RAM Logic/Buffer. The RAM Logic buffer contains the input and output buffers for data and date/time information, the logic for writing information into the RAM and reading information out of the RAM for display. The Scroll button on the card (66) addresses the locations in RAM where the selected information is stored.

63. Read-Write RAM. The read-write RAM is used to store the sensor data, patient information and ID for readout by LCD display or by the Remote Wand unit.

64. USB Port Or Other Computer Connection. The optional USB or other computer connector or port is connected to the RAM through the RAM Logic buffer to read out patient identification and sensor data. The jack can also be connected to a computer for downloading this information into the patient's database.

65. Diaper & Patient ID Push button(s) Circuit.

66. Scroll Button.

67. LCD Readout.

68. The LOGIC ASSISTED CANCEL button (68) on the card will be connected through a logic circuit that enables its use for (i) cutting off the alarm, (ii) choosing the sensor or (iii) exiting patient the ID input mode, and (iv) cancelling or exiting a scrolling operation. It will only be active for one of the three functions at a time, depending upon which function is selected by the other buttons on the card.

69. A Transmitting Antenna.

70. Sensitivity Buttons.

71. Sensor Button On Card.

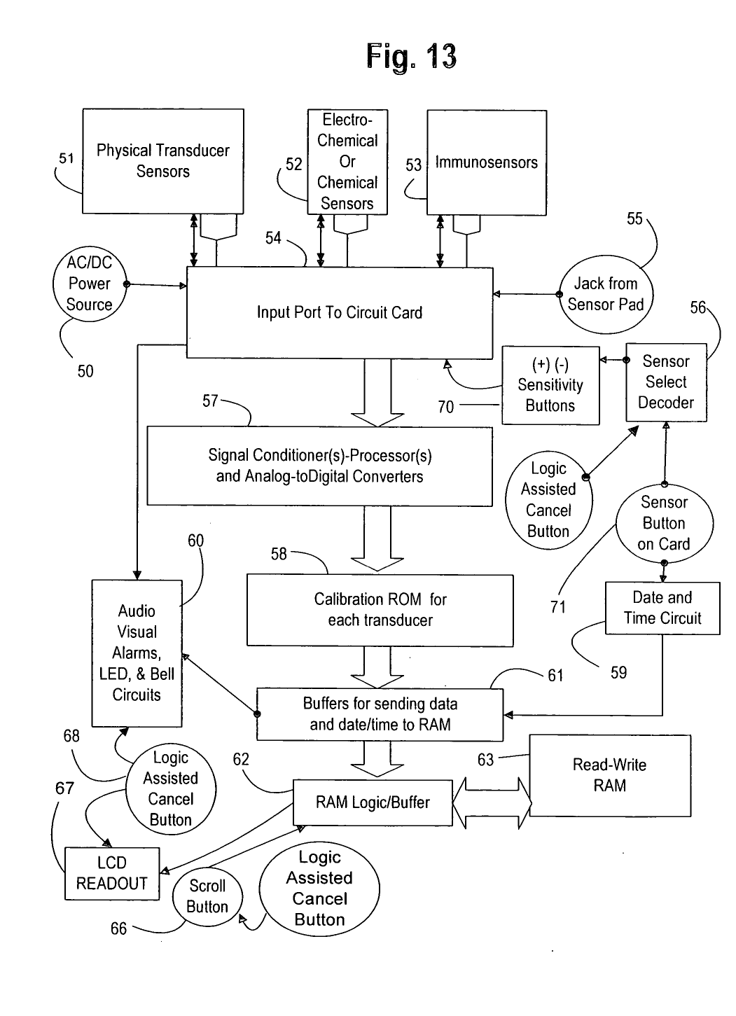

FIG. 13 depicts a more advanced reusable or disposable hygienic diaper, sensor pad, and or sensor belt system with wetness and bowel movement alert means, and for sensing and treating disease. This embodiment incorporates the following basic components: a schematic block diagram view of the “non-deluxe circuit card unit” for home use where automated readings from sensor finding(s) are not collected from multiple individuals nor transmitted through wireless means.

Normal Circuit Card for Home Use:

This card is similar to the deluxe model, with all of the components expressed and numbered as in FIG. 12, except that it does not have a transmission alarm, it does not have programmable push buttons 65, and it is shown not having an optional USB or other computer connector or port 64 as described in FIG. 12, though some models for the home may have the connector port 64 shown in FIG. 12 as well to upload sensor finding(s) to a computer for specialized home-care studies.

Controllable Functions:

The circuit card is powered by a DC battery and has the option to be plugged into an AC outlet for longer use on non-ambulatory patients. The Scroll (select source) button on the face of the card selects the transducers mounted on the diaper. The sensitivity selector on the card adjusts the gain and sensitivity of the sensors for the individual patient. The date and time are selectable by the program buttons to set the date/time circuit, which is used to annotate the data when a discharge is detected. The audio and visual alarms are also selected by the Scroll (select source) button and cleared by the LOGIC ASSISTED CANCEL button (68).

A further description of specific circuit blocks within the non-deluxe version of the home “circuit card unit” include:

Description of Specific Circuit Blocks:

50. DC power source with AC adapter—the battery is connected to the card and the AC selector is attached to a bridge-filter circuit to convert the AC voltage to a DC level suitable for operation of the circuit card.

51-53. The sensor circuits are divided into three regions to accommodate the different electronic requirements to process and decode the signals arising from each.

51. Physical transducer/sensors include variables such as temperature and humidity (wetness). They are attached to the sensitivity selector through the input port (54) that adjusts the gain on each through a potentiometer. Outputs from the sensors go to signal conditioning circuits (57), including analog-to-digital conversion. The processed signal goes to a calibration circuit (58) for conversion into digitized interpreted data for storage and transmission. A digital signal present at the signal conversion circuit output activates a series of AND gates to pass the digitized sensor data with digital date and time information to the memory. Simultaneously, an activation signal is sent to the enabled alarms.

52. Electro-chemical and or chemical sensors include variables such as sugar levels, ketone levels, blood and other chemicals related to medical conditions, or chemical warfare. They are attached to the sensitivity selector through the input port (54) that adjusts the gain on each by a potentiometer. Outputs from the sensors go to signal conditioning circuits (57), including analog-to-digital conversion. The processed signal goes to a calibration circuit (58) for conversion into interpreted data for storage and transmission. A digital signal present at the signal conversion circuit output activates a series of AND gates to pass the digitized sensor data with digital date and time information to the memory. Simultaneously, an activation signal is sent to the enabled alarms.

53. Immunosensors include variables such as bacterial and viral concentrations, toxins, parasites and emissions related to skin conditions, or to bacteriological or viral warfare. They are attached to the sensitivity selector through the input port (54) that adjusts the gain on each by a potentiometer. Outputs from the sensors go to signal conditioning circuits (57), including analog-to-digital conversion. The processed signal goes to a calibration circuit (58) for conversion into interpreted data for storage and transmission. A digital signal present at the signal conversion circuit output activates a series of AND gates to pass the digitized sensor data with digital date and time information to the memory. Simultaneously, an activation signal is sent to the enabled alarms.

54. Input Port To Circuit Card.

55. Jack From Sensor Pad. The jack from the sensor pad takes the input from the sensors on the pad to the input port using identical buffers and logic as from the sensors on the diaper.

56. Sensor Select Decoder. The decoder circuit allows scrolling through each mounted sensor. When one is selected, a flip-flop will be set to enable or disable that sensor in response to the CLEAR button that turns the selected sensor off. A timing circuit in the port allows clearing the selected sensor to be disabled within 5 seconds of selecting the sensor. Otherwise the sensor will remain active.

57. Signal Conditioner(s)-Processor(s) and Analog-to-Digital Converters. The signal conditioners and processors amplify, filter and convert the signals from the sensors to digital format. These are sent to the calibration Read-only-memory (ROM) for processing.

58. Calibration ROM For Each Transducer. The calibration Read-only-memory (ROM) contains the calibration code algorithm to convert the conditioned signal data to usable data, such as specific temperature or humidity units. This digital data can then be stored in RAM, used to set off a selected set of alarms or appear on the LCD readout on the card.

59. Date & Time Circuit. The date and time circuit can be set with the Scroll (sensor) button on the card. It contains an internal clock and buffers to store date and time digitally. These are temporarily stored in the data buffers (61) to be sent to memory or the LCD readout when sensor data are present to tag sensor data finding(s) and stamp them with a time and date along with a patient/diaper/sensor type identification/and sensor area location identification (to specify what each sensor plugged into the circuit card unit is sensing).

60. Audio-Visual Alarms, LED, Bell, & Transmitting RF Modulation Circuits. The alarm system consists of audio, visual and transmission alarms. They are activated by the Scroll (sensor) button and cleared by the CANCEL button. The audio alarm is a bell, the visual alarm is a light emitting diode (LED) and the transmission alarm is transmitted to the Nurse Remote Wand or directly to the nurses' station computer(s) by the attached antenna (69). A decoder sends the alarm information to the LCD display for readout.

61. Buffers For Sending Data And Date/Time To RAM. The data buffers collect the calibrated digital data from the Calibration ROM (58) and the Date/Time circuit (59) and send the information to the selected alarms (60), the LCD readouts (67) and the RAM buffer for storage.

62. RAM Logic/Buffer. The RAM Logic buffer contains the input and output buffers for data and date/time information, the logic for writing information into the RAM and reading information out of the RAM for display. The Scroll button on the card (66) addresses the locations in RAM where the selected information is stored.

63. Read-Write RAM. The read-write RAM is used to store the sensor data, patient information and ID for readout by LCD display or by the Remote Wand unit.

66. Scroll Button.

67. LCD Readout.

68. The LOGIC ASSISTED CANCEL button (68) on the card will be connected through a logic circuit that enables its use for (i) cutting off the alarm, (ii) choosing the sensor or (iii) exiting patient the ID input mode, and (iv) cancelling or exiting a scrolling operation. It will only be active for one of the three functions at a time, depending upon which function is selected by the other buttons on the card.

70. Sensitivity Buttons.

71. Sensor Button On Card.

The following descriptions and items relate to the “Nurse Remote Wand” Unit:

Nurse Remote Wand:

The Nurse Remote Wand has a USB port or other computer port to allow it to download or upload sensor finding(s) data received from the Circuit Card Unit(s) to computer(s) and or output collection of data received from the read-write RAM collected from the Circuit Card Unit(s).

Controllable Functions:

The Nurse Remote Wand is powered by a battery, or a DC power source, and has an AC adapter. The Scroll (select source) button and the sensitivity selector on the face of the Wand attach to the Circuit Card and scroll to select the sensors mounted on the diaper and their sensitivity. The Wand can also be used to select the date and time on the Circuit Card. The audio, visual and transmission alarms are also selected by the “Scroll Button” 19 (select source) (as is also shown in FIGS. 9, 10, and 11) and the alarms are cleared by the CANCEL button 17 (as is shown in FIGS. 9, 10 and 11). Audio, visual and transmission alarms are on the Nurse Remote Wand and are selected by the “Scroll Button” (select source) and cleared by the CANCEL button. A USB connector 22 (as shown in FIGS. 9, 10, and 11) is built into the Nurse Remote Wand for attachment to a computer for downloading of sensor and or patient information.

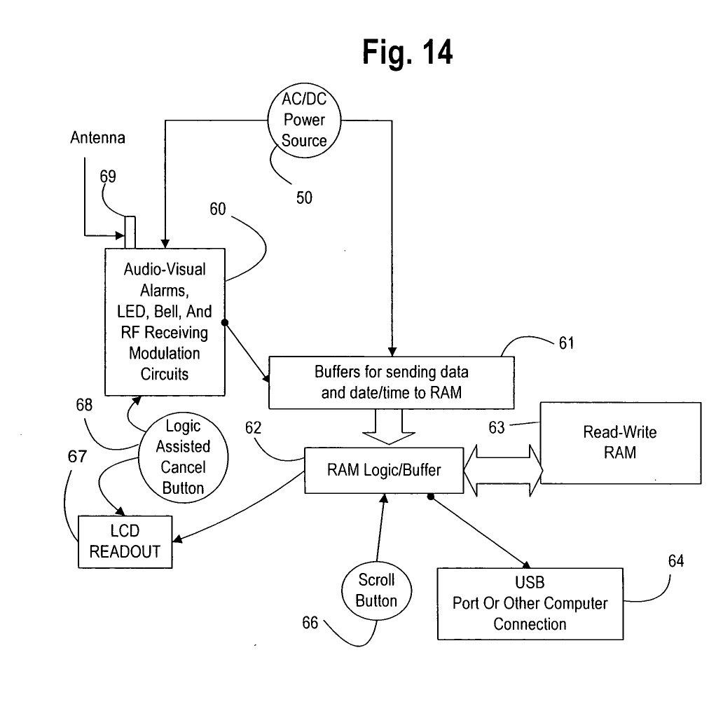

FIG. 14 depicts a schematic block diagram view of the “Nurse Remote Wand” for home use, being the non-deluxe version where automated readings from sensor finding(s) are not collected from multiple individuals nor transmitted through wireless means, however optional means can include a USB port 22 (as is also shown in FIGS. 9, 10 and 11) or another computer port that can download sensor finding(s) to a computer.

Furthermore, FIG. 14 is comprised of the following items:

50. DC power source with AC adapter—the battery is connected to the nurse remote wand and the AC selector is attached to a bridge-filter circuit to convert the AC voltage to a DC level suitable for operation of the nurse remote wand.

60. The Audio-Visual Alarms LED, Bell, And RF Receiving Modulation Circuits. Alarms are received from the Circuit Card through the antenna. The alarm system consists of audio and visual alarms, activated by the Scroll (select source) button and cleared by the CANCEL button. The audio alarm is a bell, and the visual alarm is a light emitting diode (LED). A decoder sends the alarm information to the LCD display for readout.

61. Buffers for sending data and date and time to RAM.

62. RAM Logic/Buffer.

63. The read-write RAM circuit provides the memory for writing data from the Circuit Card, temporary storage of the data and subsequent output read to a computer.

64. USB port or other computer connection with handshake interface circuits that are used to initiate communication for reading data into computer. This is attached to a direct memory access circuit (DMA) for downloading patient data onto the computer.

66. Scroll Button.

67. The decoder circuit for LCD readout translates digital sensor data into an LCD display readout.

68. The LOGIC ASSISTED CANCEL button on the card will be connected through a logic circuit that enables its use for (i) cutting off the alarm, (ii) cancelling or exiting a scrolling operation. It will only be active for any of the functions at a time, depending upon which function is selected by the other buttons on the card.

69. A Receiving Antenna.

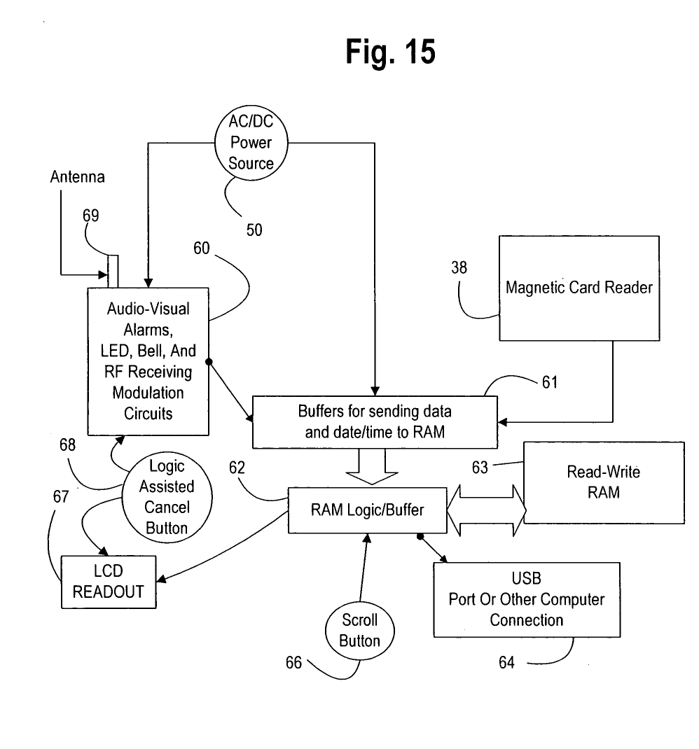

FIG. 15 depicts a schematic block diagram view of the “Nurse Remote Wand” for hospital, nursing home, or day care use, being the deluxe version where automated readings from sensor finding(s) are collected from multiple individuals and transmitted through wireless means, and where the nurse remote wand has a magnetic card reader 38 (shown in FIG. 10) built into it, having a USB port 22 (as is shown in FIGS. 9, 10, and 11) or having another computer port that can download sensor finding(s) to a computer.

Furthermore, FIG. 15 is comprised of the following items:

38. The card reader consists of a magnetic card reader and decoder to transfer data from a Magnetically-Striped Patient Identification Card 43 (shown in FIG. 10) having a readable magnetic stripe 44 (shown in FIG. 10) that contains coded patient information that will be transferred to the Nurse Remote Wand by swiping it through the magnetic card reader and then transferred subsequently to a computer.

50. DC power source with AC adapter—the battery is connected to the nurse remote wand and the AC selector is attached to a bridge-filter circuit to convert the AC voltage to a DC level suitable for operation of the nurse remote wand.

60. The Audio-Visual Alarms LED, Bell, And RF Receiving Modulation Circuits. Alarms are received from the Circuit Card through the antenna. The alarm system consists of audio and visual alarms, activated by the Scroll (select source) button and cleared by the CANCEL button. The audio alarm is a bell, and the visual alarm is a light emitting diode (LED). A decoder sends the alarm information to the LCD display for readout.

61. Buffers for sending data and date and time to RAM.

62. RAM Logic/Buffer.

63. The read-write RAM circuit provides the memory for writing data from the Circuit Card, temporary storage of the data and subsequent output read to a computer.

64. USB port or other computer connection with handshake interface circuits that are used to initiate communication for reading data into computer. This is attached to a direct memory access circuit (DMA) for downloading patient data onto the computer.

66. Scroll Button.

67. The decoder circuit for LCD readout translates digital sensor data into an LCD display readout.

68. The LOGIC ASSISTED CANCEL button on the card will be connected through a logic circuit that enables its use for (i) cutting off the alarm, (ii) cancelling or exiting a scrolling operation. It will only be active for any of the functions at a time, depending upon which function is selected by the other buttons on the card.

70. A Receiving Antenna.

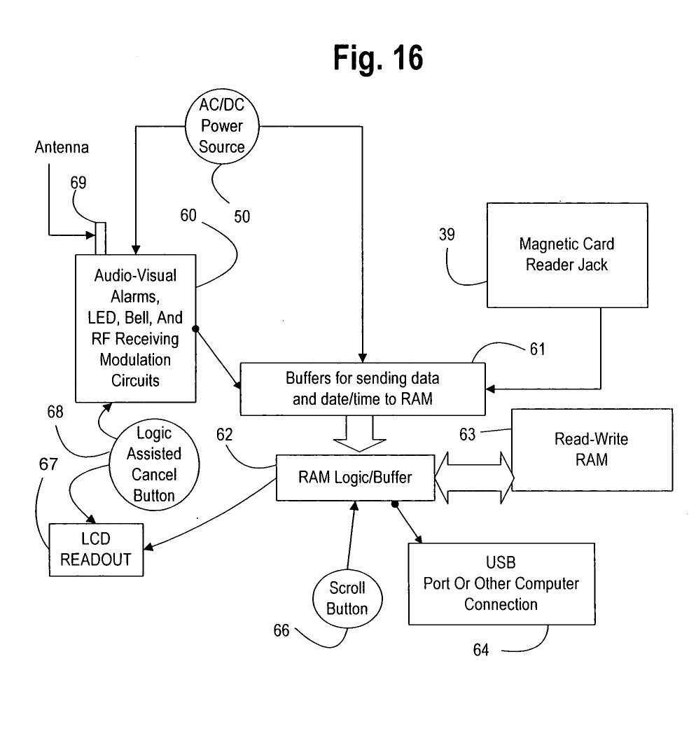

FIG. 16 depicts a schematic block diagram view of the “Nurse Remote Wand” for hospital, nursing home, or day care use, being an alternative deluxe version of the “Nurse Remote Wand” where automated readings from sensor finding(s) are collected from multiple individuals and transmitted through wireless means, and having an optional jack 39 (shown in FIG. 11) to attach an optional magnetic card reader 38 (shown in FIG. 11) to the “Nurse Remote Wand” as an accessory item; the “Nurse Remote Wand” having a USB port 22 (as is shown in FIGS. 9, 10 and 11) or having another computer port that can download sensor finding(s) to a computer.

Furthermore FIG. 16 is further comprised of the following items:

Description of Specific Circuit Blocks:

39. The Nurse Remote Wand has a Jack 39 (shown in FIGS. 11 and 16) for attaching a magnetic card reader 38 (shown in FIG. 11) that consists of a magnetic card reader and decoder to transfer data from a Magnetically-Striped Patient Identification Card 43 (shown in FIG. 10) having a readable magnetic stripe 44 (shown in FIG. 10) that contains coded patient information that will be transferred to the Nurse Remote Wand by swiping it through the magnetic card reader and then transferred subsequently to a computer.

50. DC power source with AC adapter—the battery is connected to the nurse remote wand and the AC selector is attached to a bridge-filter circuit to convert the AC voltage to a DC level suitable for operation of the nurse remote wand.

60. The Audio-Visual Alarms LED, Bell, And RF Receiving Modulation Circuits. Alarms are received from the Circuit Card through the antenna. The alarm system consists of audio and visual alarms, activated by the Scroll (select source) button and cleared by the CANCEL button. The audio alarm is a bell, and the visual alarm is a light emitting diode (LED). A decoder sends the alarm information to the LCD display for readout.

61. Buffers for sending data and date and time to RAM.

62. RAM Logic/Buffer.

63. The read-write RAM circuit provides the memory for writing data from the Circuit Card, temporary storage of the data and subsequent output read to a computer.

64. USB port or other computer connection with handshake interface circuits that are used to initiate communication for reading data into computer. This is attached to a direct memory access circuit (DMA) for downloading patient data onto the computer.

66. Scroll Button.

67. The decoder circuit for LCD readout translates digital sensor data into an LCD display readout.

68. The LOGIC ASSISTED CANCEL button on the card will be connected through a logic circuit that enables its use for (i) cutting off the alarm, (ii) cancelling or exiting a scrolling operation. It will only be active for any of the functions at a time, depending upon which function is selected by the other buttons on the card.

71. A Receiving Antenna.

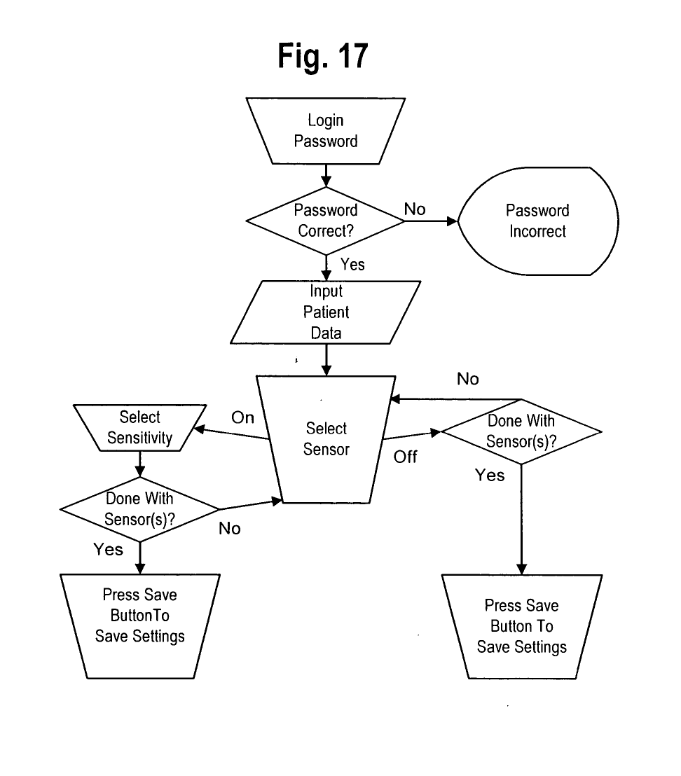

FIG. 17 depicts a software schematic block diagram illustrating various requirements and items:

Software Requirements:

1. A LOGIN procedure with password protection is used to access the Circuit Card operation and diaper and patient data (shown in FIG. 17).

2. Once the Circuit Card is accessed, setting of diaper and patient IDs is input into the read-write RAM on the Card. Sensors are selected to input data from the diaper.

3. Once the sensors have signals from patient discharge, calculation and calibration of sensor output to usable data is performed.

4. Digitized sensor data is then stored in RAM to the database and decoded to the LCD display.

5. Handshaking software is used for initiating communication with a computer.

### The Sensor Pad:

The sensor pad utilizes the same sensor circuits in the Circuit Card Unit from the diaper. The difference is that the signals are fed through the sensor pad sensor(s) and or through a connector jack 45b (as shown in FIG. 18) connecting optional sensor(s) 45c (as shown in FIG. 18) to the sensor pad 45 (as shown in FIG. 18) which in turn connect through electrodes or wires 4 (as shown in FIG. 18) to the Circuit Card Unit 7 (as shown in FIG. 18) through “electrode snaps” or “connection(s)” 2 (as shown in FIG. 18) on the Sensor Pad. It should also be noted that one or more connector(s) can be installed to accommodate optional sensor(s) on the sensor pad each having means to allow the insertion of one or more sensor(s) into each connector for sensing detection interest(s).

### The Sensor Pad Belt:

The sensor pad belt utilizes the same sensor circuits in the Circuit Card Unit from the diaper. The difference is that the signals are fed through the sensor pad sensor(s) and or through a connector jack 45b (as shown in FIG. 18A) connecting optional sensor(s) 45c (as shown in FIG. 18A) to the sensor pad belt 45 (as shown in FIG. 18A) which in turn connect through electrodes or wires 4 (as shown in FIG. 18A) to the Circuit Card Unit 7 (as shown in FIG. 18A) through a “electrode snaps” or “connection(s)” 2 (as shown in FIG. 18A) on the Sensor Pad Belt. It should also be noted that one or more connector(s) can be installed to accommodate optional sensor(s) on the sensor pad each having means to allow the insertion of one or more sensor(s) into each connector for sensing detection interest(s).

FIG. 18 is a perspective view of an alternate embodiment of the present invention showing a sensing pad 45 (as opposed to a diaper) of “X” length and “Y” width” having an absorbent layer(s) 24 embedded with one or more sensor(s) 5(a) embedded in a sensor layer 5 within absorbent layer(s) 24 and optional sensor connector(s) 45(b) positioned throughout the pad that allow additional sensors to be connected to the sensor pad through wired means so that sensing can be applied to a wound or to contaminated areas, or to spills, for sensing detection and reporting application applications the same way the diaper works to be connected through “n” number of electrode snaps 2 in communication with the “circuit card unit” 7 (as illustrated in FIGS. 1, 6, 7, and 8) through “n” number of electrodes or wires 4 shown in the pad, having optional adhesive attachment means on its edges, or other attachment means 46, to attach the pad to the skin or to an area for sensing, analyses, and reporting functions; and or where additional sensors may be connected to the sensing pad through one or more optional sensor connector(s) 45(b) each designed to insert one or more sensor leads to accommodate one or more optional sensor(s) 45(c) in them, positioned throughout the pad that allow additional sensors 45(c) to be connected to the sensor pad through wired means whereby all sensor(s) are connected through “n” number of electrodes or wires to “n” number of electrode snaps whereby the “circuit card unit” can be attached.

FIG. 18(A) is a perspective view of an alternate embodiment of the present invention showing a sensing pad 45 (as opposed to a diaper) of “X” length and “Y” width” having an absorbent layer(s) 24 embedded with one or more sensor(s) 5(a) embedded in a sensor layer 5 within absorbent layer(s) 24 that can be worn as a belt 45(a) around a body or around a wrist, or an ankle or otherwise tied to a body, or to an object, and where sensors may connect to the sensor layer 5 to also body areas, to sense contaminated areas, or spills, or for sensing detection and reporting applications the same way the diaper works to be connected through “n” number of electrode snaps 2 in communication with the “circuit card unit” 7 (as illustrated in FIGS. 1, 6, 7, and 8) through “n” number of electrodes or wires 4 shown in the pad, having optional adhesive attachment means on its edges, or other attachment means 46, to attach the pad to the skin or to an area for sensing, analyses, and reporting functions having means to provide all the functionality of the present invention of the aforementioned claims to a sensing pad (as opposed to a diaper) having “X” length and “Y” width”, or any shape and size, having absorbent layer(s) embedded with one or more sensor(s) that can be worn as a belt around a body or around a wrist, or an ankle or otherwise tied to a body, or to an object, or to contaminated areas, or to spills, for sensing, detection and reporting application applications the same way the diaper works; whereby sensor(s) are embedded in the absorbent layers of the belt and or where sensor(s) are exposed to the surface of the pad; and or where additional sensors may be connected to the sensing belt through one or more optional sensor connector(s) 45(b), each designed to insert one or more sensor leads to accommodate one or more optional sensor(s) 45(c) in them, each connector positioned throughout the belt or pad that allow additional sensors 45(c) to be connected to the sensor pad or belt through wired means whereby all sensor(s) are connected through “n” number of electrodes or wires to “n” number of electrode snaps whereby the “circuit card unit” can be attached, and whereby the pad has optional adhesive attachment means on its edges to attach the pad to the skin, or other attachment means, and whereby the pad can be applied to an area for sensing, analyses, and reporting functions.

### Further Details are as Follows:

The Outer Shell 8 Of The Disposable Diaper (Is Made Of A Plastic-Type Material In One Variation And Can Be Non-Porous Or Semi-permeable).

The Absorbent Layer(s) Of The Diaper 24. (The invention herein implies that it is understood that a diaper may be constructed with just 1 absorbent layer to a maximum of “n” layers, and that sensors may be strategically embedded where appropriate to provide the benefit intended).

The inner “absorbent layer 24” is the closest “absorbent” layer to the infant or adult wearing the diaper. It is sandwiched between the “permeable lining” and the “sensors”. This “absorbent layer is designed to “absorb” liquids and to saturate the sensors placed between the “absorbent” layers: “absorbent layer 1 and absorbent layer 2”, namely the “foil wet sensor” and “other sensors” described below”.

Foil wetness lining sensors or other sensor(s) 5 are sandwiched in absorbent layers of diaper communicating through electrodes and connected through electrode snaps connected to sensors from the small circuit card unit that snaps on to diaper. Circuit card unit senses wetness, disease, and abnormalities, and connects to sensors. Other sensors designed to detect disease and abnormalities are sandwiched between absorbent layers of the diaper.

The Circuit Card Unit Can Operate Via A Battery Or Via An AC Adapter For Hospitalized Patients Confined To Hospital Beds. The Permeable Lining Between The Infant Or Adult And “Absorbent Layer #1”, Designed To Allow Absorption Into The Absorbent Layers Of The Diapers. Nurse Remote Wand Is Used For Monitoring Deluxe Versions Of Diapers I Hospital Or Special Care Facilities Where There Are Multiple Patients And Sanitary Conditions Are To Be Observed.

The Outer Shell Of The Disposable Diaper (Is Made Of A Plastic-Type Material). The “outer shell” of the diaper is similar to other disposable diapers made out of thin plastics or nylons or other similar materials commonly found in the industry. The materials here are not the thrust of the invention. The functionality of the invention is the thrust of the invention. The “outer shell” is to provide a somewhat “water-proof” barrier between the absorbent layers that are contained within the diaper to the outside world and to minimize leakage of urine and feces from being expelled out of the diaper to keep things sanitary.

Foil wetness lining sensor or other sensor(s) 5 sandwiched between absorbent layers of diaper. (The invention herein implies that it is understood that a diaper may be constructed with just 1 absorbent layer to a maximum of “n” layers, and that sensors may be strategically embedded where appropriate to provide the benefit intended). The “foil-wet sensor is a foil-type sensor that lays in between “absorbent layer 1 and absorbent layer 2” and is designed to alert the caregiver(s) when wetness is sensed.

Though there are numerous sensors that can be used to sense wetness, a thin foil sensor or other sensor(s) 5 may be used to lay between the absorbent layers to sense the entire area where urine may be absorbed and where the foil may proceed to the waistline where it comes to contact to terminals called: “electrode snaps”, described below, that allow the circuit card unit to attach to the foil-wet sensor. Sensors can be placed in various positions throughout the diaper, either throughout the entire lining of the diaper sandwiched between the absorbent layers 1 and 2 or within an absorbent layer if there is one layer or directly under the permeable layer or in contact with the wearer of the diaper if the sensor can be woven into the diaper in a comfortable and in a hygienic fashion. Different sensors can have different uses, to sense wetness, and also to analyze vaginal or penile discharge and secretions for abnormalities, bacteria, viruses, and or disease.

Algorithms are also built into the design of the system that are utilized by logic circuits that take sensor data and analyze findings and provide data to nursing or medical staff stating what sensors have found in expelled diapers after analyzing urine, feces, or other matter, utilizing algorithms, look-up tables, and calculations and or formulas to convey findings to Nurse Remote Wand units and to patient databases.

Absorbent Layer Of Diaper. (The invention herein implies that it is understood that a diaper may be constructed with just 1 absorbent layer to a maximum of “n” layers, and that sensors may be strategically embedded where appropriate to provide the benefit intended). The “absorbent layer 2” is the furthest “absorbent” layer from the infant or adult wearing the diaper. It is sandwiched between the “sensors” and the outer shell of the diaper. The “absorbent layer is designed to “absorb” liquids and to saturate the sensors placed between the “absorbent” layers: “absorbent layer 1 and absorbent layer 2”, namely the “foil wet sensor” and “other sensors” described.

Electrode Snaps Communicate From Sensors To Small Circuit Card Unit That Snaps On To Diaper. The “electrode snaps” are terminal connection contacts that allow a caregiver to snap the “circuit card unit” off of one diaper and place it on another diaper so the caregiver can re-use the “circuit card unit” over and over again. The contacts or “electrode snaps” can be designed to accommodate different diaper designs including just a “wetness only diaper design”, “a wetness with multiple disease and abnormality diagnosing functionality capabilities diaper design”, “a wetness with single disease and or abnormality diagnosing functionality capabilities diaper designs”, “or with a targeted single disease and abnormality diagnosing functionality capability diaper design”.

Circuit Card Unit 7 Senses Wetness, Disease, And Abnormalities, And Connects To Sensors. The “circuit card unit” is a small portable removable electrical component of the diaper that connects to the sensor(s) of the diaper. It attaches to the waist area of the diaper through a snap-fit connection through the “electrode snaps” where a caregiver can snap the unit on and off from a diaper and re-use the device. It is battery powered and or powered through an AC adapter.

It has a number of buttons on it for controlling the device. One button on the deluxe models for hospitals allows a caregiver to assign a diaper ID on the circuit card that will transmit a signal to a nurse remote wand that will alert a caregiver when the diaper becomes wet, and it will tell whose diaper it is by displaying the diaper ID on the nurse remote wand so a caregiver knows what patient it is. Obviously, the “circuit card unit” will also have to have input button means to input a code diaper ID for the patient. The alert will happen when wetness occurs or when a disease or other impurity or abnormality has been sensed by the sensors depending on the diaper model used.

The circuit card unit 7 also has an LED that flashes on all models when wetness is sensed for home use, and it also has a feature that can be turned on and off to provide an audible alert by depressing a button on or off (status of the audible alert on/off can be verified by an LCD readout on the circuit card unit on the wearer of the diaper). On the deluxe model for hospitals and other facilities, the audible alert on the nurse remote wand can be turned on or off as well and there will be a visual LCD readout on the nurse remote wand for a caregiver to inspect this as well. Furthermore, there is a speaker that is built into the “circuit card unit” and in the “nurse remote wand” for audible alerts and there are bright LED visual alerts both on the “circuit card unit” and on the “nurse remote wand” as well for both wetness and or disease, bacteria, virus, and or abnormality alerts. To defeat an alert on the deluxe unit for hospitals, the “circuit card unit” has a button that should be depressed three times and within a minute the caregiver should also depress the cancel alert button on the “nurse remote wand” while that specific patient's diaper ID is showing in the LCD on the “nurse remote wand”. To defeat an alert on a home unit just depress the button three times. The structure of the “circuit card unit” is to be made as small and inexpensively as possible to fit preferably and comfortably on the waistline of the diaper when snapped on securely. The casing can be made out of plastic with the electronics inside designed to function as indicated.

The Circuit Card Unit Can Operate Via A Battery Or Via An AC Adapter For Hospitalized Patients Confined To Hospital Beds. The “power source” can be battery or via an AC adapter. Various batteries can be used, or AC adapters depending on the design parameters.

Other Sensors Designed To Detect Disease And Abnormalities Sandwiched Between Absorbent Layers Of The Diaper. (The invention herein implies that it is understood that a diaper may be constructed at least 1 absorbent layer to a maximum of “n” layers, and that sensors may be strategically embedded where appropriate to provide the benefits intended). The “other sensors” contained in the diaper are for sensing diseases, viruses, blood, bacteria, ketone levels, sugar levels, and other abnormalities found in urine, and the secretion of penile and vaginal secretions and feces, via the gasses, materials, and the liquids that will be expelled into the diaper and also to report body temperature or fever conditions.

The sensors 5 can also sense these items and help diagnose a normal condition. Sensors can be placed in various positions throughout the diaper, either throughout the entire lining of the diaper sandwiched between the absorbent layers 1 and 2, within multiple absorbent layers, or within one absorbent layer if there is one layer or directly under the permeable layer or in contact with the wearer of the diaper if the sensor can be woven into the diaper in a comfortable and in a hygienic fashion. Different sensors can have different uses, to sense wetness, and also to analyze anal, vaginal or penile discharge and secretions for abnormalities, bacteria, viruses, and or disease. Sensor(s) can be electronic or chemical. Chemical sensors can either change color or provide information when triggered.

The Permeable Lining Between The Infant Or Adult And “Absorbent Layer #1”, Designed To Allow Absorption Into The Absorbent Layers Of The Diapers. The “permeable lining” is optional and is the barrier between the absorbent layer closest to the wearer of the diaper and it allows liquids to pass through it to be absorbed into the absorbent layer(s). The “permeable lining” is a thin porous lining or membrane.

The Nurse Remote Wand 25 Is Used For Monitoring Deluxe Versions Of Diapers In Hospital Or Special Care Facilities Where There Are Multiple Patients And Sanitary Conditions Are To Be Observed. The “nurse remote wand” is a portable wand much like a remote control of a television in size designed to be a receiver for the audible and visual alerts received from the diapers when the diaper becomes wet or when a disease, bacteria, virus, blood, or other abnormality is detected. It also communicates with the diaper. The wand becomes triggered when the anal, penile or vaginal secretions or when the urine or the feces are found to have found abnormalities from the gasses or the liquids found as detected from the sensor(s). The default LCD readout is “normal”. Upon an alert, the “nurse remote wand” will provide an LCD readout with the diaper ID of the patient that needs attention, it will state whether the diaper is wet and the readout on the “nurse remote wand” will state whether a disease or other abnormality has been sensed. Furthermore, a bright LED alert will blink on the “nurse remote wand” when an alert is made and an audible alert can be selectively turned on or off on the “nurse remote wand” as well to keep caregivers on their toes. Obviously, there is a speaker built into the “nurse remote wand” for the audible alert to work.

The “nurse remote wand” works on batteries and it has a certain range measured in feet that it can work on from the patients that wear the diapers. If multiple alerts happen the “nurse remote wand” stores the next alert in memory and shows it once the first alert is cleared out of memory. The “nurse remote wand” is cleared by depressing both the “nurse remote wand” and the “circuit card unit”. To defeat an alert on the deluxe unit for hospitals, the “circuit card unit” has a button that should be depressed three times and within a minute the caregiver should also depress the cancel alert button on the “nurse remote wand” while that specific patient's diaper ID is showing in the LCD on the “nurse remote wand”. The “nurse remote wand” is similar to a television remote control device in size and shape and is designed to receive visual and audible alerts and to cancel alerts and to communicate with the “circuit card unit”. It is also battery operated.

The “circuit card unit” is a small portable removable electrical component of the diaper that connects to the sensor(s) of the diaper. It attaches to the waist area of the diaper through a snap-fit connection through the “electrode snaps” where a caregiver can snap the unit on and off from a diaper and re-use the device. It is battery powered and or powered through an AC adapter. It has a number of buttons on it for controlling the device. One button on the deluxe models for hospitals allows a caregiver to assign a diaper ID on the circuit card that will transmit a signal to a nurse remote wand that will alert a caregiver when the diaper becomes wet, and it will tell whose diaper it is by displaying the diaper ID on the nurse remote wand so a caregiver knows what patient it is. Obviously, the “circuit card unit” will also have to have input button means to input a code diaper ID for the patient.

The alert will happen when wetness occurs or when a disease or other impurity or abnormality has been sensed by the sensors depending on the diaper model used. The circuit card unit also has an LED that flashes on all models when wetness is sensed for home use, and it also has a feature that can be turned on and off to provide an audible alert by depressing a button on or off (status of the audible alert on/off can be verified by an LCD readout on the circuit card unit on the wearer of the diaper). On the deluxe model for hospitals and other facilities, the audible alert on the nurse remote wand can be turned on or off as well and there will be a visual LCD readout on the nurse remote wand for a caregiver to inspect this as well. Furthermore, there is a speaker that is built into the “circuit card unit” and in the “nurse remote wand” for audible alerts and there are bright LED visual alerts both on the “circuit card unit” and on the “nurse remote wand” as well for both wetness and or disease, bacteria, virus, and or abnormality alerts. To defeat an alert on the deluxe unit for hospitals, the “circuit card unit” has a button that should be depressed three times and within a minute the caregiver should also depress the cancel alert button on the “nurse remote wand” while that specific patient's diaper ID is showing in the LCD on the “nurse remote wand”. To defeat an alert on a home unit just depress the button three times.

The “nurse remote wand” is a portable wand much like a remote control of a television in size designed to be a receiver for the audible and visual alerts received from the diapers when the diaper becomes wet or when a disease, bacteria, virus, blood, or other abnormality is detected.

The wand becomes triggered when the anal, penile or vaginal secretions or when the urine or the feces are found to have found abnormalities from the gasses or the liquids found as detected from the sensor(s). The default LCD readout is “normal”. Upon an alert, the “nurse remote wand” will provide a LCD readout with the diaper ID of the patient that needs attention, it will state whether the diaper is wet and the readout on the “nurse remote wand” will state whether a disease or other abnormality has been sensed. Furthermore, a bright LED alert will blink on the “nurse remote wand” when an alert is made and an audible alert can be selectively turned on or off on the “nurse remote wand” as well to keep caregivers on their toes. Obviously, there is a speaker built into the “nurse remote wand” for the audible alert to work. The “nurse remote wand” works on batteries and it has a certain range measured in feet that it can work on from the patients that wear the diapers.

If multiple alerts happen the “nurse remote wand” stores the next alert in memory and shows it once the first alert is cleared out of memory.

The “nurse remote wand” is cleared by depressing both the “nurse remote wand” and the “circuit card unit”. To defeat an alert on the deluxe unit for hospitals, the “circuit card unit” has a button that should be depressed three times and within a minute the caregiver should also depress the cancel alert button on the “nurse remote wand” while that specific patient's diaper ID is showing in the LCD on the “nurse remote wand”. Though there are numerous sensors that can be used to sense wetness, a thin foil may be used to lay between the absorbent layers to sense the entire area where urine may be absorbed and where the foil may proceed to the waistline where it comes to contact to terminals called: “electrode snaps”, described below, that allow the circuit card unit to attach to the foil-wet sensor.

The “nurse remote wand” has audible and or visual alert alarm means to warn caregivers that wetness of the diaper or bowel movements have occurred whereby a user can selectively choose to select to use “audible alerts only”, “visual alerts only”, “readouts of sensors only” or a desired combination thereof for any, all, or for specific patient(s).

The “nurse remote wand” also receives and stores data from the diaper's sensors from individual patients. This data can also be downloaded to a computer either simultaneously via a remote wireless or wired link to a computer or it can be downloaded to a computer from the “nurse remote wand” after the data has been received into the patient database system. The proposed diaper system is designed to function with a software system that allows nursing staff or doctors to enter data such as patient information, progress data, notes, diagnoses, color of feces, weight of feces and or urine, consistency of urine, and to create other user definable fields in a database for patient monitoring and progress maintenance.

Sensors can be placed in various positions throughout the diaper, either throughout the entire lining of the diaper sandwiched between the absorbent layers 1 and 2, within multiple layers, or within an absorbent layer if there is one layer or directly under the permeable layer or in contact with the wearer of the diaper if the sensor can be woven into the diaper in a comfortable and in a hygienic fashion. Different sensors can have different uses, to sense wetness, and also to analyze anal, vaginal or penile discharge and secretions for abnormalities, bacteria, viruses, and or disease. The “other sensors” contained in the diaper are for sensing diseases, viruses, blood, bacteria, ketone levels, sugar levels, and other abnormalities found in urine, and the secretion of anal, penile and vaginal secretions and feces, via the gasses, materials, and the liquids that will be expelled into the diaper. The sensors can also sense these items and help diagnose a normal condition. When an infant, toddler, child, or an adult expels urine, feces, or secretions via anal, penile or vaginal excretion and it is expelled into the diaper the sensors placed in the diaper pick up on the wetness.

Depending on the diaper model and the sensors used in the diaper various functionalities can be performed.

Wetness and other elements can be sensed, including but not limited to blood, sugar levels, ketone levels, bacteria, viruses, impurities, and other abnormalities such as to report body temperature or fever conditions, that sensor information can pick up through liquid, materials, and gas analysis from items that are expelled into the diaper.

As already described, a diaper with one or more absorbent layers is proposed with inexpensive disposable embedded sensors throughout the diaper and other diapers can be constructed where a diaper may be opened and sensors can be inserted for one-time use or where re-useable sensor(s) can be used. Again, chemical and electronic sensors can be used and combined in one diaper or different models can be made for different uses. The sensors can be continuous so as to be like a thin sheet of foil sandwiched between two or more layers of absorbent layers within the diaper or embedded within one layer of a one-layered absorbent diaper. The sensor(s) can be varied to sense either wetness only and create one model of diaper, or many diaper models can be made to sense diseases, bacteria, viruses, impurities, blood, ketone levels, sugar levels, and other abnormalities as well. Of course, various models can be marketed, one may be wetness only to detect whether urination has occurred, and or usually followed with feces expulsion, or other diaper models can be developed and marketed targeted to diagnose and sense for different conditions and diseases each to be manufactured with different sensors. The diapers can also be used in facilities as a method for stool collection.

When an infant, toddler, child, or an adult expels urine, feces, or secretions via anal, penile or vaginal excretion and it is expelled into the diaper the sensors placed in the diaper pick up on the wetness. Depending on the diaper model and the sensors used in the diaper various functionalities can be performed. Wetness and other elements can be sensed, including but not limited to blood, sugar levels, ketone levels, bacteria, viruses, impurities, and other abnormalities that sensor information can pick up through liquid and gas analysis from items that are expelled into the diaper. Once the diaper absorbs what is expelled into it the sensor(s) in the diaper pick up information and relay that information to the “circuit card unit”.

Sensor operational information contains the following: Though there are numerous sensors that can be used to sense wetness, a thin foil may be used to lay between the absorbent layers to sense the entire area where urine may be absorbed and where the foil may proceed to the waistline where it comes to contact to terminals called: “electrode snaps”, (described below), that allow the circuit card unit to attach to the foil-wet sensor. Sensors can be placed in various positions throughout the diaper, either throughout the entire lining of the diaper sandwiched between the absorbent layers 1 and 2, within multiple layers, or within an absorbent layer if there is one layer or directly under the permeable layer or in contact with the wearer of the diaper if the sensor can be woven into the diaper in a comfortable and in a hygienic fashion. Different sensors can have different uses, to sense wetness, and also to analyze vaginal or penile discharge and secretions for abnormalities, bacteria, viruses, and or disease.

The “other sensors” contained in the diaper are for sensing diseases, viruses, blood, bacteria, ketone levels, sugar levels, and other abnormalities found in urine, and the secretion of anal, penile and vaginal secretions urine, feces, and other matter via the gasses and the liquids that will be expelled into the diaper. The sensors can also sense these items and help diagnose a normal condition. After the sensors pick up their information the information is transmitted to the “circuit card unit” whose operational information is as follows:

The “circuit card unit” is a small portable removable electrical component of the diaper that connects to the sensor(s) of the diaper. It attaches to the waist area of the diaper through a snap-fit connection through the “electrode snaps” where a caregiver can snap the unit on and off from a diaper and re-use the device. It is battery powered and or powered through an AC adapter. It has a number of buttons on it for controlling the device. One button on the deluxe models for hospitals allows a caregiver to assign a diaper ID on the circuit card that will transmit a signal to a nurse remote wand that will alert a caregiver when the diaper becomes wet, and it will tell whose diaper it is by displaying the diaper ID on the nurse remote wand so a caregiver knows what patient it is.

The “circuit card unit” as does the “nurse remote wand” also has audible and or visual alert alarm means to warn caregivers that wetness of the diaper or bowel movements have occurred whereby a user can selectively choose to select to use “audible alerts only”, “visual alerts only”, “readouts of sensors only” or a desired combination thereof for any, all, or for specific patient(s).

Obviously, the “circuit card unit” will also have to have input button means to input a code diaper ID for the patient. The alert will happen when wetness occurs or when a disease or other impurity or abnormality has been sensed by the sensors depending on the diaper model used.

The circuit card unit also has an LED that flashes on all models when wetness is sensed for home use, and it also has a feature that can be turned on and off to provide an audible alert by depressing a button on or off (status of the audible alert on/off can be verified by an LCD readout on the circuit card unit on the wearer of the diaper). On the deluxe model for hospitals and other facilities, the audible alert on the nurse remote wand can be turned on or off as well and there will be a visual LCD readout on the nurse remote wand for a caregiver to inspect this as well. Furthermore, there is a speaker that is built into the “circuit card unit” and in the “nurse remote wand” for audible alerts and there are bright LED visual alerts both on the “circuit card unit” and on the “nurse remote wand” as well for both wetness and or disease, bacteria, virus, and or abnormality alerts. To defeat an alert on the deluxe unit for hospitals, the “circuit card unit” has a button that should be depressed three times and within a minute the caregiver should also depress the cancel alert button on the “nurse remote wand” while that specific patient's diaper ID is showing in the LCD on the “nurse remote wand”.

To defeat an alert on a home unit just depress the button three times. It is contemplated that the circuit card unit will have the ability to analyze sensor information and display the appropriate warning(s) as to what it has sensed on a LCD readout and to provide an audible and a bright LED visual alert. In addition, it will give these alerts to a remote “nurse remote wand” through radio transmission means.

All circuit means, including diaper ID information for multiple patient monitoring will be transmitted to the “nurse remote wands” in the deluxe models. Input means on the “circuit control units” will have the ability to add diaper ID information for each diaper. In addition, the circuit card units will have memory to store the diaper ID information and the appropriate buttons to scroll through alert warnings and to cancel alerts and to turn the unit on or off and to clear its memory.

In addition, the circuit card unit will have all the integrated circuits; wiring; a battery compartment; an AC adapter connection; a speaker for audible alerts; a Light Emitting Diode for visual alerts; a Liquid Crystal Display for wetness alerts where the words: “DIAPER WET” or another warning can be displayed when wetness is sensed; the default display on the Liquid Crystal Display would always read: “Diaper Dry”; the same Liquid Crystal Display can be used for displaying diseases or codes for such diseases and for abnormalities that are sensed from the secretions and or the gasses and the liquids that are expelled into the diaper from anal, penile, and vaginal secretions; connections and or terminal snap-fit connections to the electrode snaps and or separately to electronic sensors; buttons for programming the diaper ID on deluxe models; an antennae for transmitting the diaper ID to “nurse remote wands” on deluxe models; scrolling features to scroll through different warnings displayed on the unit (these warnings can range from wetness to a range of diseases or abnormalities sensed from the sensors utilized); a cancel button to cancel the alert; a power button to turn the circuit card unit on or off and to clear the circuit card unit's memory from the warnings and the alerts.

The circuit card unit will have all the electrical components required for the intended for the operations as herein described within this document in all its various models and can be constructed differently in different models having more and or less features to accommodate more or less functionalities with variations as desired depending on the sensors utilized and the functionalities desired. It is contemplated that the home units will not require transmission means to be built into the circuit card unit where transmission will be sent to a “nurse remote wand” and many of the features on the “deluxe circuit card unit” will not be required for home units. Features not required on home units would be those where programmability for Diaper IDs would be available. The home unit “circuit card unit” shows the non-deluxe version as depicted in the drawings. The “circuit card unit” will also allow a diaper to be constructed that will alert caregivers that disease and or other abnormalities that exist in the urine, in secretions or in the feces through circuit and sensor means and that will allow the clocking of the actual time when wetness or secretions are found by sensors or when anal, penile or vaginal secretions are expelled into the diaper and have been sensed by the circuit card unit. It is obvious by this functionality that a clock circuit will be included in the circuit card unit that keeps track of the time for this feature that will store the actual time of such expulsion(s) into the memory of the circuit card unit of the diaper and that a caregiver can scroll on the “circuit card unit” through the messages on the “LCD” display to check for the time of expulsion into the diaper.

In the deluxe version of the “circuit card units” for the hospital units, the caregiver can see this information on the “nurse remote wand” as well because the “actual time” will be transmitted immediately once wetness, secretion, disease identification, abnormality identification, and or expulsion occurs, and the “circuit card unit” will also display the same information that is attached to the diaper of the patient. On home units no “nurse remote wands” are used, but caregiver(s) can scroll through messages on the LCD to view the actual time of wetness, secretion, disease identification, abnormality identification, and or expulsion(s) as home units still have the same functionalities except for the “nurse remote wand” capability.

Alerts on the deluxe versions for hospitals are controlled through the “nurse remote wand” which is the size and shape of a television remote designed to communicate with the diaper circuit card units whose operational information is as follows: The “nurse remote wand” is a portable wand much like a remote control of a television in size designed to be a receiver for the audible and visual alerts received from the diapers when the diaper becomes wet or when a disease, bacteria, virus, blood, or other abnormality is detected. The wand becomes triggered when the penile or vaginal secretions or when the urine or the feces are found to have found abnormalities from the gasses or the liquids found as detected from the sensor(s).

The default LCD readout is “normal”. Upon an alert, the “nurse remote wand” will provide a LCD readout with the diaper ID of the patient that needs attention, it will state whether the diaper is wet and the readout on the “nurse remote wand” will state whether a disease or other abnormality has been sensed. Furthermore, a bright LED alert will blink on the “nurse remote wand” when an alert is made and an audible alert can be selectively turned on or off on the “nurse remote wand” as well to keep caregivers on their toes. Obviously, there is a speaker built into the “nurse remote wand” for the audible alert to work. The “nurse remote wand” works on batteries and it has a certain range measured in feet that it can work on from the patients that wear the diapers. If multiple alerts happen the “nurse remote wand” stores the next alert in memory and shows it once the first alert is cleared out of memory. The “nurse remote wand” is cleared by depressing both the “nurse remote wand” and the “circuit card unit”. To defeat an alert on the deluxe unit for hospitals, the “circuit card unit” has a button that should be depressed three times and within a minute the caregiver should also depress the cancel alert button on the “nurse remote wand” while that specific patient's diaper ID is showing in the LCD on the “nurse remote wand”. The deluxe versions of the hygienic diaper, that implement the use of the “nurse remote wand” will allow the dumping of patient data from the “nurse remote wand” into computer files, databases, and for hard copy computer printouts on paper for the deluxe hospital models using the “nurse remote wands”. The “nurse remote wands” can be connected to a computer. The patient data can be transferred to a computer database and printouts can be printed on all the patients for progress reports that will give caregiver(s) and doctors information on the actual time of wetness, secretion, disease identification, abnormality identification, and or expulsion(s) and it will identify the condition(s) sensed by the sensor(s) and provide that information to the caregiver(s) and the doctor(s) in report form.

The system will also come with software that will enable doctors(s), nurses and caregiver(s) to weigh a clean diaper and weigh a full diaper after expulsion(s) and or secretion(s) have been expelled or secreted into the diaper so that information can also be inputted into the record for each patient. The Software provided will work automatically to combine information with a patient's Diaper ID number or identification that was programmed by caregivers and update records as time goes on with each diaper reset and cancellation when a new clean diaper is put on a patient.

Graphing functionalities for the actual time of wetness, secretion, disease identification, abnormality identification, and or to account for actual time(s) of expulsion(s) into the diaper are built into the software as well as graphing functionalities for keeping track of the weight of a full diaper once matter has been expelled into the diaper, whether it is anal, penile, vaginal secretions or expulsions. Other features of the software will allow one to record the color of anal, penile, and vaginal secretion(s) and expulsion(s), their consistency, whether the patient had loose stools or diarrhea, and how the patient is responding to medication and to track progress and the success of medical treatment as well as what is being used for the medical treatment, type of drug(s), vaccines being used and dosage(s) and doctors and nurses assigned to each patient as well as other information including, patient name, patient ID, social security number, diagnostic history, medical history database information, insurance company of individual, referrer, and other information built into the software that can be accessed also in graphical form.

There is also a section where one can record notes for each patient in the record for each patient for each diaper change to track progress.

Also, the diaper will have medication(s), salves, ointments, powder(s), aloe, healing agents, or other lotions chemically added to the diaper in the absorbent layers or to other layer(s) of the diaper that come into contact with the wearer of the diaper for the treatment of diseases, abnormalities and or condition(s) associated with diaper rashes and or other diseases and condition(s) and abnormalities that can be treated by the wearing of a diaper through contact means or through transdermal transmission from the diaper to the skin.

Furthermore, the diaper may be used as a method to collect stool, urine, or other materials in various alternate models of the invention within the spirit of a system for the treatment of disease and as stated in the provisional case, whereby a sealable watertight “tear-off stool-urine-materials-collection pouch” is provided, along with an area on the pouch for marking the pouch with patient data, a pouch having watertight sealable closure means to protect stool, materials, or urine samples, as shown in the drawings as an alternate feature whereas the pouch can be built in and attached to the diaper through attachment means and included somewhere on the exterior or in the interior of the diaper for easy removal by a caregiver, to be detached and used for biological collection and analysis and sealed in a sealable sanitary fashion and marked for use by laboratories.

As to a further discussion of the manner of usage and operation of the present invention, the same should be apparent from the above description. Accordingly, no further discussion relating to the manner of usage and operation will be provided.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

What is claimed is:

1. A more hygienic diaper, sensor pad, and or sensing belt with alert, readout, transmission, paging, software and patient information database recording means for treating and caring for wetness, feces, and disease comprising:
a) a disposable or re-useable diaper having a wetness alert system and porous material within the body of the diaper containing sensors for detecting and analyzing wetness or the contents of a full diaper including the skin condition of the wearer and or to analyze and detect materials, gasses and or liquids expelled into the diaper;
b) a diaper, sensor pad, or sensor belt with a porous inner lining, encased by a non-permeable or a semi-permeable outer lining with one or more electronic, electrochemical, or chemical sensors positioned throughout the diaper to sense wetness, medical conditions, drugs, or other variables;
c) a diaper, sensor pad or sensor belt with one or more removable and or reusable or disposable “sensors” placed and attached either with adhesive(s), velcro, snaps, or other attachment means, within absorbent layers of the diaper or to touch the skin in communication with the diaper, and or to insert into body cavities such as the anal, vaginal and penile canals, to detect and analyze and report body temperature, skin condition, ketone levels, sugar levels, blood, body fluids, liquids, gasses, drugs, bacteria, viruses, toxins, diseases, parasites, viruses, etc., or other agents that can be detected by sensor means and to display said findings detected from sensor(s) on readouts on the “circuit card unit(s)” and on the “nurse remote wand(s);
i) certain diaper models also have temperature sensors or sensor probes for disease that are securely held in position on the skin and or in the anal, vaginal or penile canals for better temperature or disease, toxin or other readings by means of the user wearing the diaper, whereby wearing the diaper the user is prevented from ejecting the sensor(s) from the skin, and or from anal, vaginal, or penile canal(s) from involuntary or voluntary muscle contractions;
d) a reusable or disposable “circuit card unit” releasably attachable and connected to the diaper and connected to sensors in the diaper with a power source to drive circuits to alert caregivers when diaper becomes wet or when there is a bowel movement; said “circuit card unit” further contains and is comprised of:
i) scrollable functions on the “circuit card unit” and a user interface to allow scrolling means to view “read-outs” on a LCD or LED readout of sensor findings on the “circuit card unit”;
ii) a user interface on the “circuit card unit” to be “reset” for the next alert;
iii) memory on the “circuit card unit” to store sensor findings;
iv) a user interface on the “circuit card unit” that will allow the “circuit card unit” to be reset for viewing the next wetness or full diaper alert and sensor finding readouts and to allow nursing or medical staff to enter, program and or display a patient ID into the “circuit card unit that can later be transmitted to the “Nurse Remote Wand” as specified in claim 1;
v) a user interface on the “circuit card unit” that will allow the “circuit card unit” to be “reset” for the next wetness or full diaper alert and sensor finding readouts for home models not having transmission needs or patient ID entering needs thereby omitting the need for having buttons on the circuit card unit for entering patient ID information;
vi) a transmitting circuit and an RF modulating circuit with an antennae on deluxe hospital units that will send sensor findings from the “circuit card unit” to either the “Nurse Remote Wand” or directly to a computer database on a separate computer or programmable digital computer through wired or wireless means;
vii) an internal clock to store in memory when wetness or material such as feces or other agents were expelled or detected by sensor(s) and to report the same on the “circuit card unit's” readouts or to transmit said information to the “Nurse Remote Wand” or to computer database(s) through wired or wireless means on the deluxe “circuit card unit” models and push button or other means to cause the circuit card unit to go into a mode to program the internal clock of the “circuit card unit” whereby the user can enter the current time and date and specify “A.M.” or “P.M.” utilizing the patient identification push buttons to enter said time and date information;
viii) a speaker for audible alerts and or a LED or LCD or other visual indicator for wetness or full diaper alerts, and readout means for sensor findings;
ix) means to stop the audio-visual alert on the “circuit card unit” by resetting the unit by depressing a button on the “circuit card unit”.
x) means to clear the memory of the “circuit card unit” to purge sensor data from all sensor findings data received from sensor(s).
xi) means to have sensor signals adjusted through circuitry in the “circuit card unit” to adjust their sensitivity to be more or less sensitive through adjustment means on the “circuit card unit” by applying amplification or resistance to the signals as desired through control means that a caregiver can adjust through selective control means, a dial, or buttons on the “circuit control card”.
xii) means to provide a “circuit card unit” to be placed on the diaper to connect to the sensor(s) of the diaper that will be water-resistant or water-proof connecting and functioning with the sensors so that it will not short circuit and that it will continue to operate even when a diaper becomes wet.
xiii) means to provide a “circuit card unit” to be placed on the diaper that will be small and flexible for the comfort and safety of the wearer.
xiv) means to provide a “circuit card unit” and sensors that will track the frequency and time of gas discharge and report the same via sensor(s) either through sensors that can sense gas discharge through chemical analyses through audible means, or through sensor(s) designed to detect gas through other means.
xv) pre-programmed means through hard-wired circuitry in the circuit card unit having the functionality to be able to turn sensor(s) on or off selectively on the circuit card unit(s) to pinpoint what area(s) on the diaper or the sensor pad should be sensed by depressing “sensor select” button to select a desired sensor, whereby a desired sensor is selected and a sensor location is displayed on the circuit card unit's readout, and where depressing the “cancel button” within a time frame will toggle that sensor on or off; and where the circuit card unit will automatically identify the sensor(s) with an area ID so when they report their data they will also report their sensor location, as their sensor ID, and or the area they are sensing to help assist data gathering information and analyses needs whereby this feature shall apply to all embodiments of the present invention and models of the deluxe “circuit card unit” for the diaper version of the present invention and to the “sensing pad” and or to the “sensor belt” versions of the present invention (shown in FIGS. 18 and 18A) by depressing a button to select sensor(s). For each diaper or sensor pad there is a Master ID for each Diaper and or Sensor Pad versions of the present invention whereby the type and location of each sensor are programmed into the attached circuit card unit allowing not only sensor reading findings data but also location data of the sensor and the type of sensor being used as information to be provided with each transmission of the data event. Each sensor and each location on the diaper or sensor pad will have its own ID and or code and these will be coded into the master ID of the diaper or sensor pad;
xvi) means for the “circuit card unit” to receive sensor information from the diaper, from the sensor pad, or from the “sensor belt”, the circuit card unit further having a wired or wireless port to connect to a computer to download its “sensor findings and clocked information”, patient identification, sensor type identification; sensor area location(s) information; diaper, sensor pad identification, and or sensor belt identification to a computer or to the “nurse remote wand” either through a wireless or through a wired connection either through a USB connection or through other wired or wireless connection means;
e) a circuit and an antennae in the circuit card unit that will allow transmission means in the circuit system of the diaper to transfer sensor data findings and alerts of wetness and bowel movements to caregivers or medical staff;
f) audible and or visual alert alarm means to warn caregivers that wetness of the diaper or bowel movements have occurred on the diaper “circuit card unit” system itself whereby a user can selectively choose to select to use “audible alerts only”, “visual alerts only”, “readouts of sensors only” for the patients or a desired combination thereof;
g) audible and or visual alert alarm means to warn caregivers that wetness of the diaper or bowel movements have occurred on the “Nurse Remote Wand” unit(s)” whereby a user can selectively choose to select to use “audible alerts only”, “visual alerts only”, “readouts of sensors only” or a desired combination thereof for any, all, or for specific patient(s);
h) algorithm(s) to identify what sensors discover and report it to the caregivers on the “Nurse Remote Wand” and or directly to wireless receiver links to computer database(s); and further, algorithm(s) to report concentration(s) of item(s) sensed by sensor(s) in the diaper(s);
i) a software interface to allow staff to input data relating to treatment plans, diagnoses, notes, and to track patient data which is editable and which has multiple security and access levels;
j) a software and a database system where sensor information on each patient is dumped and stored from transmission(s) sent from diaper(s);
k) a battery operated portable “Nurse Remote Wand”, with circuitry to accommodate rechargeable and regular batteries, that receives sensor data through transmission means from the diaper's circuit system which also allows the data it receives to be downloaded through a port to a computer patient treatment, patient progress, diagnosis, and planning database; said “Nurse Remote Wand” further contains and is comprised of:
i) scrollable functions on the “Nurse Remote Wand” and a user interface to allow scrolling means to view “read-outs” on a LCD or LED readout of sensor findings on the “Nurse Remote Wand”;
ii) a user interface on the “Nurse Remote Wand” to be “reset” for the next alert;
iii) a memory circuit on the “Nurse Remote Wand” to store sensor findings for optional transmission of sensor data or uploading to a computer;
iv) a user interface on the “Nurse Remote Wand” that will allow the “Nurse Remote Wand” to be reset for the next wetness or full diaper alert and or to view sensor finding readouts;
v) a transmitting receiving circuit and antennae on the “Nurse Remote Wand” on deluxe hospital units that will receive sensor findings from the “circuit card unit” and or a receiving circuit designed to interface to a computer through a port to connect to a computer database on a separate computer or on a programmable digital computer through wired or wireless means;
vi) means for the “Nurse Remote Wand” to receive sensor information from the “circuit card unit” on the diaper and a wired or wireless port to connect to a computer and dump its “sensor findings and clocked information” to a computer either through wireless or wired connection(s) either through a USB connection or through other wired or wireless connection means;
vii) means to stop the audio-visual alert on the “Nurse Remote Wand” by resetting the unit by depressing a button on the “Nurse Remote Wand”;
viii) means to clear the memory of the “Nurse Remote Wand” to purge sensor data from all sensor findings data tables stored on the “Nurse Remote Wand” received from circuit card unit transmission(s);
ix) means to provide a “nurse remote wand” with an optional “magnetic card reader” built into it for reading patient identification cards containing medical information that can be stored into the “nurse remote wand” and also later downloaded to a computer to a patient database along with sensor information picked up from diaper sensor(s);
x) means to provide a “nurse remote wand” with an optional “magnetic card reader” jack built into it for attaching a separate “magnetic card reader” to the “nurse remote wand” for reading patient identification cards containing medical information that can be stored into the “nurse remote wand” and also later downloaded to a computer to a patient database along with sensor information picked up from diaper sensor(s).
l) an audio-visual alarm and indicator on the “circuit card unit” for wetness and bowel movement alerts, and a LCD or LED or other readout means to display what sensors have detected, and a “battery” or other power source to drive the “circuit card unit” on the diaper to apply safe low voltage to drive sensor, analysis, alert and transmission circuits, with the circuitry to allow the use of regular and rechargeable batteries;
m) an audio-visual alarm and indicator on the “Nurse Remote Wand” for wetness and bowel movement alerts, and a LCD or LED or other readout means to display what sensors have detected, and a “battery” or other power source to drive the “Nurse Remote Wand” unit circuits;
n) a LED or LCD or other readout means to convey what diseases, toxins, ketone levels, sugar levels, bacteria types, viruses, drugs, or other abnormalities that the sensors have found to caregivers or to otherwise report a healthy condition;
o) a diaper with an attached and sanitary sealable watertight “tear-off stool-urine-materials collection pouch” that can be torn off, or detached from the diaper and used for stool, urine or materials collection purposes, and made for specimen collection purposes and for refrigeration and to withstand temperatures required in analysis, having a sealable water-tight sanitary closure means to seal urine, blood, secretions, body fluids, loose or solid stools, or other biological samples and to prevent leakage, also having an area upon the pouch that can be written upon and marked with patient data identification and laboratory information and or notes, to be incorporated in optional various models of the subject invention for medical collection and laboratory analysis purposes;
p) a software and database system to allow caregivers and or medical staff to input and edit individual patient data; to input and edit diagnoses; to input and edit treatment plans; to view edit or change notes to sensor findings; to view or enter notes; to graph progress; to view, edit, add or change fields in the database, to print reports, and to allow the automatic receipt of data from the “Nurse Remote Wand” into the database relating to patient sensor data from received diaper transmission(s), and
q) “electrode snaps” and or other connection means to connect sensors to the “circuit card unit” of the diaper;

2. A more hygienic diaper, sensor pad, and or sensing belt with alert, readout, transmission, paging, software and patient information database recording means for treating and caring for wetness, feces, and disease comprising:
a) one or more sensor(s) comprising one or more biosensor(s) having at least one immunosensor, or other types of inorganic electronic, electrochemical, or chemical sensors; the sensor(s) being adapted to detect and or analyze bodily waste contamination or other variables that can be sensed such that might be of interest such as: drugs, toxins, bacteria, blood, blood sugar levels, ketone levels, organisms, pathogens, viruses, parasites, disease, gasses, liquids, body fluids, such as may be present in urine or in matter expelled into the diaper from skin, penile, or vaginal secretions, or fecal material on surface(s) contacted by the diaper in communication with said sensor(s) and to provide a signal to indicating the presence of the waste contamination on the surface wherein the surface is skin;
b) a diaper wetness alert system with a portable “Nurse Remote Wand” as claimed in claim 1 that can receive wetness and full diaper alerts and readouts containing sensor findings for treating wetness and diseases that nurses and doctors can carry around as a portable device or keep handy at nursing stations to monitor the condition of patients;
c) a diaper wetness alert system with a portable “Nurse Remote Wand” as claimed in claim 1 that can receive wetness and full diaper alerts and readouts containing sensor findings for treating wetness and diseases, and so that caregivers can change diapers promptly to treat patients with rashes, skin conditions, diarrhea, diseases and problems associated with urine and fecal expulsion;
d) a diaper wetness alert system with a portable “Nurse Remote Wand” as claimed in claim 1 that can receive wetness and full diaper alerts and readouts containing sensor findings for treating wetness and diseases that can download sensor findings to a patient database.

3. A more hygienic diaper, sensor pad, and or sensing belt with alert, readout, transmission, paging, software and patient information database recording means for treating and caring for wetness, feces, and disease comprising:
a) a diaper system that can use reusable or disposable sensor(s) for detecting and analyzing specific desired medical conditions or analysis needs where sensors are in communication with the “circuit card unit” in claim 1 and the matter expelled in the diaper and the patient's skin whereby the sensor(s) can be one or more in number and can be mixed in number in the diaper to detect and analyze a variety of condition(s), agents, or variables that staff might be interested in, and where sensors may be removed or changed on the diaper through “electrode snaps” or connections as specified in claim 1 to accommodate any sensor desired for targeting and accommodating desired sensing needs;
b) means to provide a diaper with one or more sensor(s) to sense, detect and analyze and report on one or more variables, such as disease, drugs, antibodies, blood, proteins, wetness, bowels, urine, ketone levels, sugar levels, parasites, bacteria, viruses, sebum, secretions, body fluids, gasses, liquids, materials, toxins, or any item that might be of interest that sensor(s) might detect including item(s) such as concentrations as sensors may be designed to sense;
c) means to strategically attach and position removable and or adjustable anal, vaginal, and penile temperature thermometers and or disease sensor(s) or probes to the diaper so they will be easily inserted into the anal, vaginal and penile canals through flexibly fitting and positional sensors to a patient in fixed and or adjustable strategic position(s) with flexibility having at least three comfortable flexible sensor(s): an anal sensor of “x” length and “y” thickness, a vaginal sensor of “x” length and “y” thickness, and or a penile sensor of “x” length and “y” thickness [where “x” and “y” can be the same and or different number(s) for any and all sensor(s)], to be positioned and inserted into anal, vaginal, and penile canals respectively, where upon the wearer being fit with the diaper with sensors in place wearing the diaper prevents the sensors from being expelled from voluntary or involuntary muscular contraction(s) by virtue of holding the sensors securely into place after insertion by the patient wearing the diaper;
d) means where removable and adjustably positional anal, vaginal, and penile sensor(s) all have their anchor socket “heads” secured, stitched and or fabricated between two absorbent layers comprised of fibrous material sandwiched between absorbent layer(s) of the diaper with socket(s) or “heads” residing in a groove cut out of the absorbent fibrous material of the diaper in the middle layer where the sockets or “heads” rest in a groove cut out in the middle absorbent layer of diaper to accommodate the size of the socket heads (to prevent the socket(s) from escaping into the rest of the diaper), with the diaper having an upper absorbent layer above each grooved area with a smaller groove opening so as to only allow the sensor probe stem to protrude from the groove; a middle absorbent layer having a groove cut out area that is larger than the groove cut out area of the upper absorbent layer to accommodate the socket “heads”, and an optional lower absorbent layer underneath the middle absorbent layer to provide a floor for the socket “heads” to travel on; with the sensing probe portion of the sensor(s) protruding inwardly towards the patient through the absorbent layers of the diaper through open groove(s) so they can be inserted into the anal, vaginal or penile canal(s), and whereby the anchor socket head(s) of the sensor(s) where said sensor(s) probe stems connect into the sockets and where the sensors can slide bi-directionally in a either a continuous groove or in separate grooves cut out in absorbent layers of the diaper whereby the sensors may slide bi-directionally within said groove(s) (so as not to allow the sensor(s) to escape into the rest of the diaper) within the absorbent layer(s) of the diaper to comfortably adjust and correctly position and insert the sensor(s) into the anal, vaginal, and penile canals respectively in a comfortably fitting manner for different size requirements of specific individuals, and whereby the heads of the sensor(s) are large enough and tight-fitting enough within said groove(s) that once adjusted they remain in their adjusted position(s) and where the sensor(s) remain supported in their upright and or in their functional flexible position(s) for a comfortable fit or insertion into their anal, vaginal or penile canals for temperature, disease or other detection interest(s); and where the anal, vaginal, and penile sensor probes may be plugged into or removed from a socket or wire connection within the groove(s) in a secure fashion through attachment means having electronic connectivity, said sockets having electrodes or wires connecting to electrode or wire connections to other sensors within the sensor layer or directly to the electrode or wire connections leading to connection(s) or “electrode snaps” on the outer portion of the diaper where the circuit card unit may be attached to said electrodes to communicate with said sensor(s); and whereby in the case of the penile sensor, the penile sensor probe may have its distance extended from the diaper by utilizing a probe having a wired connection of “x” length connecting the penile sensor probe to the diaper at a socket or a wire contact location where the penile sensor can be attached to the diaper and where the penile sensor may then be placed manually into the penile canal to accommodate the easy placement of the penile sensor into the penile canal; and where the wired version of the penile sensor connector can also be removed or detached from the socket as desired and replaced with other types of sensor(s) with extendable wires such that can be run and placed unto different parts of the body to measure different things such as respiration, heartbeat, pulse, skin condition, bleeding, sinus trouble or wheezing, or other detection needs as mentioned, or as may be desired. Whereas anal, vaginal and penile sensor(s) can also be designed to operate in alternate models of the diaper it may be fashioned to have a plurality of sensor(s) or one or more of such sensors in addition to the anal, vaginal, or penile sensor(s) or in lieu of anal, vaginal or penile sensor(s) or a diaper may be designed to be incorporated in absence of said anal, vaginal or penile sensor(s) where there are “x” number of sensor(s) with extendable wires connected to the diaper that can be used for detection requirements;
e) means to provide a strap-on diaper with built-in attached strategically placed sensors designed for anal, vaginal or penile canal readings whereby a patient is prevented from expelling the sensor(s) from said canals through voluntary or through involuntary muscular contractions because the diaper holds the sensor(s) in their inserted position(s) as the diaper is strapped on the patient or the wearer;
f) a signal processor connected to the readout means for processing the output electrical signals from the readout means, wherein the sensor detecting and analyzing means comprise a singular sensor or an array or sensor(s) or a plurality of sensor(s) placed strategically within the diaper's absorbent layers, or to come into contact with the skin of the diaper wearer, or to be placed within anal, vaginal, or penile canals for sensing functions, wherein each detecting sensor element detects and converts signals received to electrical signals that can be processed and be transmitted to yield valuable information from sensors that can be stored, displayed and or transmitted and or downloaded or uploaded into a “Nurse Remote Wand” or directly to a computer or to the circuit card unit's readout.
g) means to provide a either one or more connector(s) on either the diaper, the sensor pad or a sensor belt type system of the present invention that will allow a caregiver or a user to connect additional or optional sensor(s) to either the diaper, the sensor pad or to the sensor belt system(s) respectively, said connector(s) to have connection means to allow the user to connect either one or more sensor(s) to each connector.

4. An alternative embodiment of the present machine, system, or device for hygiene and care requirements providing a Sensing Pad (As Opposed To A Diaper) With Alert Means For Treating And Caring For Wetness, Feces, & Disease” comprising:
a) Means to provide all the functionality of the present invention of the aforementioned claims to a sensing pad (as opposed to a diaper) having “X” length and “Y” width”, or any shape and size, having absorbent layer(s) embedded with one or more sensor(s) that can be applied to a wound, to contaminated areas or to spills, for sensing, detection and reporting application applications the same way the diaper works; whereby sensor(s) are embedded in the absorbent layers and or where sensor(s) are exposed to the surface of the pad and are connected through “n” number of electrodes or wires to “n” number of electrode snaps whereby the “circuit card unit” can be attached, and whereby the pad has optional adhesive attachment means on its edges to attach the pad to the skin, or other attachment means, and whereby the pad can be applied to an area for sensing, analyses, and reporting functions.
b) means to provide all the functionality of the present invention of the aforementioned claims to a sensing pad (as opposed to a diaper) having “X” length and “Y” width”, or any shape and size, having absorbent layer(s) embedded with one or more sensor(s) that can be worn as a belt around a body or around a wrist, or an ankle or otherwise tied to a body, or to an object, or to contaminated areas, or to spills, for sensing, detection and reporting application applications the same way the diaper works; whereby sensor(s) are embedded in the absorbent layers of the belt and or where sensor(s) are exposed to the surface of the pad and are connected through “n” number of electrodes or wires to “n” number of electrode snaps whereby the “circuit card unit” can be attached, and whereby the pad has optional adhesive attachment means on its edges to attach the pad to the skin, or other attachment means, and whereby the pad can be applied to an area for sensing, analyses, and reporting functions.
